SEQUENCE LISTING (179 sequences)

SEQ ID NO: 1 - DNA, 1131 bp, Homo sapiens
  TADG-16 cDNA sequence
ggggcgccccgggcccggcgcgagaggaggcagagggggcgtcaggccgcgggagaggaggccatgggcgcgcgcggggcgctgctgctggcgctgctgctggctcgggctggactcaggaagccggagtcgcaggaggcggcgccgttatcaggaccatgcggccgacgggtcatcacgtcgcgcatcgtgggtggagaggacgccgaactcgggcgttggccgtggcaggggagcctgcgcctgtgggattcccacgtatgcggagtgagcctgctcagccaccgctgggcactcacggcggcgcactgctttgaaacgtatagtgaccttagtgatccctccgggtggatggtccagtttggccagctgacttccatgccatccttctggagcctgcaggcctactacacccgttacttcgtatcgaatatctatctgagccctcgctacctggggaattcaccctatgacattgccttggtgaagctgtctgcacctgtcacctacactaaacacatccagcccatctgtctccaggcctccacatttgagtttgagaaccggacagactgctgggtgactggctgggggtacatcaaagaggatgaggcactgccatctccccacaccctccaggaagttcaggtcgccatcataaacaactctatgtgcaaccacctcttcctcaagtacagtttccgcaaggacatctttggagacatggtttgtgctggcaatgcccaaggcgggaaggatgcctgcttcggtgactcaggtggacccttggcctgtaacaagaatggactgtggtatcagattggagtcgtgagctggggagtgggctgtggtcggcccaatcggcccggtgtctacaccaatatcagccaccactttgagtggatccagaagctgatggcccagagtggcatgtcccagccagacccctcctggccgctactctttttccctcttctctgggctctcccactcctggggccggtctgagcctacctgagcccatgcagcctggggccaactgccaagtcaggccctggttctcttctgtcttgtttggtaataaacacattccagttgatgccttgcagggcattcttcaaaaaaaaaaaa

SEQ ID NO: 2 - protein, 314 aa, Homo sapiens
  TADG-16 protein sequence
MGARGALLLALLLARAGLRKPESQEAAPLSGPCGRRVITSRIVGGEDAELGRWPWQGSLRLWDSHVCGVSLLSHRWALTAAHCFETYSDLSDPSGWMVQFGQLTSMPSFWSLQAYYTRYFVSNIYLSPRYLGNSPYDIALVKLSAPVTYTKHIQPICLQASTFEFENRTDCWVTGWGYIKEDEALPSPHTLQEVQVAIINNSMCNHLFLKYSFRKDIFGDMVCAGNAQGGKDACFGDSGGPLACNKNGLWYQIGVVSWGVGCGRPNRPGVYTNISHHFEWIQKLMAQSGMSQPDPSWPLLFFPLLWALPLLGPV

SEQ ID NO: 3 - protein, 244 aa, Unknown
  PEPTIDE: Sequence of Protease M
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 4 - protein, 247 aa, Unknown
  PEPTIDE: Sequence of Trypsinogen
MNPLLILTFVAAALAAPFDDDDKIVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 5 - protein, 253 aa, Unknown
  PEPTIDE: Sequence of SCCE
MARSLLLPLQILLLSLALETAGEEAQGDKIIDGAPCARGSHPWQVALLSGNQLHCGGVLVNERWVLTAAHCKMNEYTVHLGSDTLGDRRAQRIKASKSFRHPGYSTQTHVNDLMLVKLNSQARLSSMVKKVRLPSRCEPPGTTCTVSGWGTTTSPDVTFPSDLMCVDVKLISPQDCTKVYKDLLENSMLCAGIPDSKKNACNGDSGGPLVCRGTLQGLVSWGTFPCGQPNDPGVYTQVCKFTKWINDTMKKHR

SEQ ID NO: 6 - protein, 421 aa, Unknown
  PEPTIDE 1, 2, 3, 4: Sequence of Hepsin, Xaa = Unknown at 1, 2, 3, 4
XXXXMAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 7 - protein, 294 aa, Homo sapiens
  PEPTIDE: TADG-16 protein sequence
MGARGALLLALLLARAGLRKPTIRGPCGRRVITSRIVGGEDAELGRWPWQGSLRLWDSHVCGVSLLSHRWALTAAHCFETYSDLSDPSGWMVQFGQLTSMPSFWSLQAYYTRYFVSNIYLSPRYLGNSPYDIALVKLSAPVTYTKHIQPICLQASTFEFENRTDCWVTGWGYIKEDEALPSPHTLQEVQVAIINNSMCNHLFLKYSFRKDIFGDMGDSGGPLACNKNGLWYQIGVVSWGVGCGRPNRPGVYTNISHHFEWIQKLMAQSGMSQPDPSWPLLFFPLLWALPLLGPV

SEQ ID NO: 8 - DNA, 23 bp, Artificial sequence
  primer_bind 6, 9, 12, 15, 18: Degenerate oligonucleotide sense primer to amplify serine proteases, n = inosine at 6, 9, 12, 15, 18
tgggtngtnacngcngcncaytg

SEQ ID NO: 9 - DNA, 20 bp, Artificial sequence
  primer_bind 3, 6, 9, 12, 18: Degenerate oligonucleotide antisense primer to amplify serine proteases, n = inosine at 3, 6, 9, 12, 18
arnggnccnccnswrtcncc

SEQ ID NO: 10 - DNA, 20 bp, Artificial sequence
  primer_bind: Oligonucleotide sense primer specific for TADG-16
gtcaggccgcgggagaggag

SEQ ID NO: 11 - DNA, 20 bp, Artificial sequence
  primer_bind: Oligonucleotide antisense primer specific for TADG-16
actctgggccatcagcttct

SEQ ID NO: 12 - DNA, 28 bp, Artificial sequence
  primer_bind: Oligonucleotide antisense primer specific for TADG-16
cggagggatcactaaggtcactatacgt

SEQ ID NO: 13 - DNA, 28 bp, Artificial sequence
  primer_bind: Oligonucleotide antisense primer specific for TADG-16
tatacgtttcaaagcagtgcgccgccgt

SEQ ID NO: 14 - DNA, 20 bp, Artificial sequence
  primer_bind: Oligonucleotide sense primer specific for TADG-16
ggtcgccatcataaacaact

SEQ ID NO: 15 - DNA, 20 bp, Artificial sequence
  primer_bind: Oligonucleotide antisense primer specific for TADG-16
actctgggccatcagcttct

SEQ ID NO: 16 - RNA, 1131 nt, Artificial sequence
  Antisense transcript of TADG-16
uuuuuuuuuuuugaagaaugcccugcaaggcaucaacuggaauguguuuauuaccaaacaagacagaagagaaccagggccugacuuggcaguuggccccaggcugcaugggcucagguaggcucagaccggccccaggagugggagagcccagagaagagggaaaaagaguagcggccaggaggggucuggcugggacaugccacucugggccaucagcuucuggauccacucaaagugguggcugauauugguguagacaccgggccgauugggccgaccacagcccacuccccagcucacgacuccaaucugauaccacaguccauucuuguuacaggccaaggguccaccugagucaccgaagcaggcauccuucccgccuugggcauugccagcacaaaccaugucuccaaagauguccuugcggaaacuguacuugaggaagaggugguugcacauagaguuguuuaugauggcgaccugaacuuccuggaggguguggggagauggcagugccucauccucuuugauguacccccagccagucacccagcagucuguccgguucucaaacucaaauguggaggccuggagacagaugggcuggauguguuuaguguaggugacaggugcagacagcuucaccaaggcaaugucauagggugaauuccccagguagcgagggcucagauagauauucgauacgaaguaacggguguaguaggccugcaggcuccagaaggauggcauggaagucagcuggccaaacuggaccauccacccggagggaucacuaaggucacuauacguuucaaagcagugcgccgccgugagugcccagcgguggcugagcaggcucacuccgcauacgugggaaucccacaggcgcaggcuccccugccacggccaacgcccgaguucggcguccucuccacccacgaugcgcgacgugaugacccgucggccgcaugguccugauaacggcgccgccuccugcgacuccggcuuccugaguccagcccgagccagcagcagcgccagcagcagcgccccgcgcgcgcccauggccuccucucccgcggccugacgcccccucugccuccucucgcgccgggcccggggcgcccc

SEQ ID NO: 17 - protein, 9 aa, Homo sapiens
  CHAIN: Residues 70-78 of the TADG-16 protein
SLLSHRWAL

SEQ ID NO: 18 - protein, 9 aa, Homo sapiens
  CHAIN: Residues 299-307 of the TADG-16 protein
LLFFPLLWA

SEQ ID NO: 19 - protein, 9 aa, Homo sapiens
  CHAIN: Residues 142-150 of the TADG-16 protein
KLSAPVTYT

SEQ ID NO: 20 - protein, 9 aa, Homo sapiens
  CHAIN: Residues 96-104 of the TADG-16 protein
WMVQFGQLT

SEQ ID NO: 21 - protein, 9 aa, Homo sapiens
  CHAIN: Residues 10-18 of the TADG-16 protein
ALLLARAGL

SEQ ID NO: 22 - protein, 9 aa, Homo sapiens
  CHAIN: Residues 252-260 of the TADG-16 protein
QIGVVSWGV

SEQ ID NO: 23 - protein, 9 aa, Homo sapiens
  CHAIN: Residues 248-256 of the TADG-16 protein
GLWYQIGVV

SEQ ID NO: 24 - protein, 9 aa, Homo sapiens
  CHAIN: Residues 139-147 of the TADG-16 protein
ALVKLSAPV

SEQ ID NO: 25 - protein, 9 aa, Homo sapiens
  CHAIN: Residues 291-299 of the TADG-16 protein
SQPDPSWPL

SEQ ID NO: 26 - protein, 9 aa, Homo sapiens
  CHAIN: Residues 130-138 of the TADG-16 protein
YLGNSPYDI

SEQ ID NO: 27 - protein, 9 aa, Homo sapiens
  CHAIN: Residues 190-198 of the TADG-16 protein
TLQEVQVAI

SEQ ID NO: 28 - protein, 9 aa, Homo sapiens
  CHAIN: Residues 6-14 of the TADG-16 protein
ALLLALLLA

SEQ ID NO: 29 - protein, 9 aa, Homo sapiens
  CHAIN: Residues 165-173 of the TADG-16 protein
FENRTDCWV

SEQ ID NO: 30 - protein, 9 aa, Homo sapiens
  CHAIN: Residues 71-79 of the TADG-16 protein
LLSHRWALT

SEQ ID NO: 31 - protein, 9 aa, Homo sapiens
  CHAIN: Residues 8-16 of the TADG-16 protein
LLALLLARA

SEQ ID NO: 32 - protein, 9 aa, Homo sapiens
  CHAIN: Residues 297-305 of the TADG-16 protein
WPLLFFPLL

SEQ ID NO: 33 - protein, 9 aa, Homo sapiens
  CHAIN: Residues 113-121 of the TADG-16 protein
QAYYTRYFV

SEQ ID NO: 34 - protein, 9 aa, Homo sapiens
  CHAIN: Residues 123-131 of the TADG-16 protein
NIYLSPRYL

SEQ ID NO: 35 - protein, 9 aa, Homo sapiens
  CHAIN: Residues 104-112 of the TADG-16 protein
TSMPSFWSL

SEQ ID NO: 36 - protein, 9 aa, Homo sapiens
  CHAIN: Residues 273-281 of the TADG-16 protein
NISHHFEWI

SEQ ID NO: 37 - protein, 9 aa, Homo sapiens
  CHAIN: Residues 70-78 of the TADG-16 protein
SLLSHRWAL

SEQ ID NO: 38 - protein, 9 aa, Homo sapiens
  CHAIN: Residues 42-50 of the TADG-16 protein
IVGGEDAEL

SEQ ID NO: 39 - protein, 9 aa, Homo sapiens
  CHAIN: Residues 10-18 of the TADG-16 protein
ALLLARAGL

SEQ ID NO: 40 - protein, 9 aa, Homo sapiens
  CHAIN: Residues 291-299 of the TADG-16 protein
SQPDPSWPL

SEQ ID NO: 41 - protein, 9 aa, Homo sapiens
  CHAIN: Residues 297-305 of the TADG-16 protein
WPLLFFPLL

SEQ ID NO: 42 - protein, 9 aa, Homo sapiens
  CHAIN: Residues 248-256 of the TADG-16 protein
GLWYQIGVV

SEQ ID NO: 43 - protein, 9 aa, Homo sapiens
  CHAIN: Residues 82-90 of the TADG-16 protein
HCFETYSDL

SEQ ID NO: 44 - protein, 9 aa, Homo sapiens
  CHAIN: Residues 142-150 of the TADG-16 protein
KLSAPVTYT

SEQ ID NO: 45 - protein, 9 aa, Homo sapiens
  CHAIN: Residues 96-104 of the TADG-16 protein
WMVQFGQLT

SEQ ID NO: 46 - protein, 9 aa, Homo sapiens
  CHAIN: Residues 299-307 of the TADG-16 protein
LLFFPLLWA

SEQ ID NO: 47 - protein, 9 aa, Homo sapiens
  CHAIN: Residues 303-311 of the TADG-16 protein
PLLWALPLL

SEQ ID NO: 48 - protein, 9 aa, Homo sapiens
  CHAIN: Residues 123-131 of the TADG-16 protein
NIYLSPRYL

SEQ ID NO: 49 - protein, 9 aa, Homo sapiens
  CHAIN: Residues 98-106 of the TADG-16 protein
VQFGQLTSM

SEQ ID NO: 50 - protein, 9 aa, Homo sapiens
  CHAIN: Residues 306-314 of the TADG-16 protein
WALPLLGPV

SEQ ID NO: 51 - protein, 9 aa, Homo sapiens
  CHAIN: Residues 71-79 of the TADG-16 protein
LLSHRWALT

SEQ ID NO: 52 - protein, 9 aa, Homo sapiens
  CHAIN: Residues 53-61 of the TADG-16 protein
WPWQGSLRL

SEQ ID NO: 53 - protein, 9 aa, Homo sapiens
  CHAIN: Residues 302-310 of the TADG-16 protein
FPLLWALPL

SEQ ID NO: 54 - protein, 9 aa, Homo sapiens
  CHAIN: Residues 130-138 of the TADG-16 protein
YLGNSPYDI

SEQ ID NO: 55 - protein, 9 aa, Homo sapiens
  CHAIN: Residues 6-14 of the TADG-16 protein
ALLLALLLA

SEQ ID NO: 56 - protein, 9 aa, Homo sapiens
  CHAIN: Residues 190-198 of the TADG-16 protein
TLQEVQVAI

SEQ ID NO: 57 - protein, 9 aa, Homo sapiens
  CHAIN: Residues 44-52 of the TADG-16 protein
GGEDAELGR

SEQ ID NO: 58 - protein, 9 aa, Homo sapiens
  CHAIN: Residues 90-98 of the TADG-16 protein
LSDPSGWMV

SEQ ID NO: 59 - protein, 9 aa, Homo sapiens
  CHAIN: Residues 143-151 of the TADG-16 protein
LSAPVTYTK

SEQ ID NO: 60 - protein, 9 aa, Homo sapiens
  CHAIN: Residues 292-300 of the TADG-16 protein
QPDPSWPLL

SEQ ID NO: 61 - protein, 9 aa, Homo sapiens
  CHAIN: Residues 203-211 of the TADG-16 protein
MCNHLFLKY

SEQ ID NO: 62 - protein, 9 aa, Homo sapiens
  CHAIN: Residues 87-95 of the TADG-16 protein
YSDLSDPSG

SEQ ID NO: 63 - protein, 9 aa, Homo sapiens
  CHAIN: Residues 168-176 of the TADG-16 protein
RTDCWVTGW

SEQ ID NO: 64 - protein, 9 aa, Homo sapiens
  CHAIN: Residues 47-55 of the TADG-16 protein
DAELGRWPW

SEQ ID NO: 65 - protein, 9 aa, Homo sapiens
  CHAIN: Residues 23-31 of the TADG-16 protein
SQEAAPLSG

SEQ ID NO: 66 - protein, 9 aa, Homo sapiens
  CHAIN: Residues 7-15 of the TADG-16 protein
LLLALLLAR

SEQ ID NO: 67 - protein, 9 aa, Homo sapiens
  CHAIN: Residues 157-165 of the TADG-16 protein
CLQASTFEF

SEQ ID NO: 68 - protein, 9 aa, Homo sapiens
  CHAIN: Residues 202-210 of the TADG-16 protein
SMCNHLFLK

SEQ ID NO: 69 - protein, 9 aa, Homo sapiens
  Residues 111-119 of the TADG-16 protein
SLQAYYTRY

SEQ ID NO: 70 - protein, 9 aa, Homo sapiens
  Residues 125-133 of the TADG-16 protein
YLSPRYLGN

SEQ ID NO: 71 - protein, 9 aa, Homo sapiens
  Residues 152-160 of the TADG-16 protein
HIQPICLQA

SEQ ID NO: 72 - protein, 9 aa, Homo sapiens
  Residues 79-87 of the TADG-16 protein
TAAHCFETY

SEQ ID NO: 73 - protein, 9 aa, Homo sapiens
  Residues 238-246 of the TADG-16 protein
SGGPLACNK

SEQ ID NO: 74 - protein, 9 aa, Homo sapiens
  Residues 172-180 of the TADG-16 protein
WVTGWGYIK

SEQ ID NO: 75 - protein, 9 aa, Homo sapiens
  Residues 110-118 of the TADG-16 protein
WSLQAYYTR

SEQ ID NO: 76 - protein, 9 aa, Homo sapiens
  Residues 191-199 of the TADG-16 protein
LQEVQVAII

SEQ ID NO: 77 - protein, 9 aa, Homo sapiens
  Residues 118-126 of the TADG-16 protein
RYFVSNIYL

SEQ ID NO: 78 - protein, 9 aa, Homo sapiens
  Residues 177-185 of the TADG-16 protein
GYIKEDEAL

SEQ ID NO: 79 - protein, 9 aa, Homo sapiens
  Residues 210-218 of the TADG-16 protein
KYSFRKDIF

SEQ ID NO: 80 - protein, 9 aa, Homo sapiens
  Residues 270-278 of the TADG-16 protein
VYTNISHHF

SEQ ID NO: 81 - protein, 9 aa, Homo sapiens
  Residues 148-156 of the TADG-16 protein
TYTKHIQPI

SEQ ID NO: 82 - protein, 9 aa, Homo sapiens
  Residues 300-308 of the TADG-16 protein
LFFPLLWAL

SEQ ID NO: 83 - protein, 9 aa, Homo sapiens
  Residues 234-242 of the TADG-16 protein
CFGDSGGPL

SEQ ID NO: 84 - protein, 9 aa, Homo sapiens
  Residues 135-143 of the TADG-16 protein
PYDIALVKL

SEQ ID NO: 85 - protein, 9 aa, Homo sapiens
  Residues 4-12 of the TADG-16 protein
RGALLLALL

SEQ ID NO: 86 - protein, 9 aa, Homo sapiens
  Residues 104-112 of the TADG-16 protein
TSMPSFWSL

SEQ ID NO: 87 - protein, 9 aa, Homo sapiens
  Residues 296-304 of the TADG-16 protein
SWPLLFFPL

SEQ ID NO: 88 - protein, 9 aa, Homo sapiens
  Residues 250-258 of the TADG-16 protein
WYQIGVVSW

SEQ ID NO: 89 - protein, 9 aa, Homo sapiens
  Residues 5-13 of the TADG-16 protein
GALLLALLL

SEQ ID NO: 90 - protein, 9 aa, Homo sapiens
  Residues 95-103 of the TADG-16 protein
GWMVQFGQL

SEQ ID NO: 91 - protein, 9 aa, Homo sapiens
  Residues 199-207 of the TADG-16 protein
INNSMCNHL

SEQ ID NO: 92 - protein, 9 aa, Homo sapiens
  Residues 297-305 of the TADG-16 protein
WPLLFFPLL

SEQ ID NO: 93 - protein, 9 aa, Homo sapiens
  Residues 291-299 of the TADG-16 protein
SQPDPSWPL

SEQ ID NO: 94 - protein, 9 aa, Homo sapiens
  Residues 183-191 of the TADG-16 protein
EALPSPHTL

SEQ ID NO: 95 - protein, 9 aa, Homo sapiens
  Residues 86-94 of the TADG-16 protein
TYSDLSDPS

SEQ ID NO: 96 - protein, 9 aa, Homo sapiens
  Residues 10-18 of the TADG-16 protein
ALLLARAGL

SEQ ID NO: 97 - protein, 9 aa, Homo sapiens
  Residues 297-305 of the TADG-16 protein
WPLLFFPLL

SEQ ID NO: 98 - protein, 9 aa, Homo sapiens
  Residues 302-310 of the TADG-16 protein
FPLLWALPL

SEQ ID NO: 99 - protein, 9 aa, Homo sapiens
  Residues 53-61 of the TADG-16 protein
WPWQGSLRL

SEQ ID NO: 100 - protein, 9 aa, Homo sapiens
  Residues 292-300 of the TADG-16 protein
QPDPSWPLL

SEQ ID NO: 101 - protein, 9 aa, Homo sapiens
  Residues 145-153 of the TADG-16 protein
APVTYTKHI

SEQ ID NO: 102 - protein, 9 aa, Homo sapiens
  Residues 42-50 of the TADG-16 protein
IVGGEDAEL

SEQ ID NO: 103 - protein, 9 aa, Homo sapiens
  Residues 10-18 of the TADG-16 protein
ALLLARAGL

SEQ ID NO: 104 - protein, 9 aa, Homo sapiens
  Residues 104-112 of the TADG-16 protein
TSMPSFWSL

SEQ ID NO: 105 - protein, 9 aa, Homo sapiens
  Residues 183-191 of the TADG-16 protein
EALPSPHTL

SEQ ID NO: 106 - protein, 9 aa, Homo sapiens
  Residues 201-209 of the TADG-16 protein
NSMCNHLFL

SEQ ID NO: 107 - protein, 9 aa, Homo sapiens
  Residues 5-13 of the TADG-16 protein
GALLLALLL

SEQ ID NO: 108 - protein, 9 aa, Homo sapiens
  Residues 291-299 of the TADG-16 protein
SQPDPSWPL

SEQ ID NO: 109 - protein, 9 aa, Homo sapiens
  Residues 70-78 of the TADG-16 protein
SLLSHRWAL

SEQ ID NO: 110 - protein, 9 aa, Homo sapiens
  Residues 195-203 of the TADG-16 protein
QVAIINNSM

SEQ ID NO: 111 - protein, 9 aa, Homo sapiens
  Residues 116-124 of the TADG-16 protein
YTRYFVSNI

SEQ ID NO: 112 - protein, 9 aa, Homo sapiens
  Residues 199-207 of the TADG-16 protein
INNSMCNHL

SEQ ID NO: 113 - protein, 9 aa, Homo sapiens
  Residues 82-90 of the TADG-16 protein
HCFETYSDL

SEQ ID NO: 114 - protein, 9 aa, Homo sapiens
  Residues 132-140 of the TADG-16 protein
GNSPYDIAL

SEQ ID NO: 115 - protein, 9 aa, Homo sapiens
  Residues 1-9 of the TADG-16 protein
MGARGALLL

SEQ ID NO: 116 - protein, 9 aa, Homo sapiens
  Residues 63-71 of the TADG-16 protein
DSHVCGVSL

SEQ ID NO: 117 - protein, 9 aa, Homo sapiens
  Residues 183-191 of the TADG-16 protein
EALPSPHTL

SEQ ID NO: 118 - protein, 9 aa, Homo sapiens
  Residues 58-66 of the TADG-16 protein
SLRLWDSHV

SEQ ID NO: 119 - protein, 9 aa, Homo sapiens
  Residues 82-90 of the TADG-16 protein
HCFETYSDL

SEQ ID NO: 120 - protein, 9 aa, Homo sapiens
  Residues 116-124 of the TADG-16 protein
YTRYFVSNI

SEQ ID NO: 121 - protein, 9 aa, Homo sapiens
  Residues 2-10 of the TADG-16 protein
GARGALLLA

SEQ ID NO: 122 - protein, 9 aa, Homo sapiens
  Residues 302-310 of the TADG-16 protein
FPLLWALPL

SEQ ID NO: 123 - protein, 9 aa, Homo sapiens
  Residues 53-61 of the TADG-16 protein
WPWQGSLRL

SEQ ID NO: 124 - protein, 9 aa, Homo sapiens
  Residues 31-39 of the TADG-16 protein
GPCGRRVIT

SEQ ID NO: 125 - protein, 9 aa, Homo sapiens
  Residues 297-305 of the TADG-16 protein
WPLLFFPLL

SEQ ID NO: 126 - protein, 9 aa, Homo sapiens
  Residues 5-13 of the TADG-16 protein
GALLLALLL

SEQ ID NO: 127 - protein, 9 aa, Homo sapiens
  Residues 71-79 of the TADG-16 protein
LLSHRWALT

SEQ ID NO: 128 - protein, 9 aa, Homo sapiens
  Residues 242-250 of the TADG-16 protein
LACNKNGLW

SEQ ID NO: 129 - protein, 9 aa, Homo sapiens
  Residues 10-18 of the TADG-16 protein
ALLLARAGL

SEQ ID NO: 130 - protein, 9 aa, Homo sapiens
  Residues 70-78 of the TADG-16 protein
SLLSHRWAL

SEQ ID NO: 131 - protein, 9 aa, Homo sapiens
  Residues 63-71 of the TADG-16 protein
DSHVCGVSL

SEQ ID NO: 132 - protein, 9 aa, Homo sapiens
  Residues 89-97 of the TADG-16 protein
DLSDPSGWM

SEQ ID NO: 133 - protein, 9 aa, Homo sapiens
  Residues 132-140 of the TADG-16 protein
GNSPYDIAL

SEQ ID NO: 134 - protein, 9 aa, Homo sapiens
  Residues 140-148 of the TADG-16 protein
LVKLSAPVT

SEQ ID NO: 135 - protein, 9 aa, Homo sapiens
  Residues 149-157 of the TADG-16 protein
YTKHIQPIC

SEQ ID NO: 136 - protein, 9 aa, Homo sapiens
  Residues 15-23 of the TADG-16 protein
RAGLRKPES

SEQ ID NO: 137 - protein, 9 aa, Homo sapiens
  Residues 117-125 of the TADG-16 protein
TRYFVSNIY

SEQ ID NO: 138 - protein, 9 aa, Homo sapiens
  Residues 51-59 of the TADG-16 protein
GRWPWQGSL

SEQ ID NO: 139 - protein, 9 aa, Homo sapiens
  Residues 263-271 of the TADG-16 protein
GRPNRPGVY

SEQ ID NO: 140 - protein, 9 aa, Homo sapiens
  Residues 74-82 of the TADG-16 protein
HRWALTAAH

SEQ ID NO: 141 - protein, 9 aa, Homo sapiens
  Residues 128-136 of the TADG-16 protein
PRYLGNSPY

SEQ ID NO: 142 - protein, 9 aa, Homo sapiens
  Residues 266-274 of the TADG-16 protein
NRPGVYTNI

SEQ ID NO: 143 - protein, 9 aa, Homo sapiens
  Residues 3-11 of the TADG-16 protein
ARGALLLAL

SEQ ID NO: 144 - protein, 9 aa, Homo sapiens
  Residues 34-42 of the TADG-16 protein
GRRVITSRI

SEQ ID NO: 145 - protein, 9 aa, Homo sapiens
  Residues 213-221 of the TADG-16 protein
FRKDIFGDM

SEQ ID NO: 146 - protein, 9 aa, Homo sapiens
  Residues 18-26 of the TADG-16 protein
LRKPESQEA

SEQ ID NO: 147 - protein, 9 aa, Homo sapiens
  Residues 101-109 of the TADG-16 protein
GQLTSMPSF

SEQ ID NO: 148 - protein, 9 aa, Homo sapiens
  Residues 227-235 of the TADG-16 protein
AQGGKDACF

SEQ ID NO: 149 - protein, 9 aa, Homo sapiens
  Residues 59-67 of the TADG-16 protein
LRLWDSHVC

SEQ ID NO: 150 - protein, 9 aa, Homo sapiens
  Residues 40-48 of the TADG-16 protein
SRIVGGEDA

SEQ ID NO: 151 - protein, 9 aa, Homo sapiens
  Residues 35-63 of the TADG-16 protein
RRVITSRIV

SEQ ID NO: 152 - protein, 9 aa, Homo sapiens
  Residues 98-106 of the TADG-16 protein
VQFGQLTSM

SEQ ID NO: 153 - protein, 9 aa, Homo sapiens
  Residues 112-120 of the TADG-16 protein
LQAYYTRYF

SEQ ID NO: 154 - protein, 9 aa, Homo sapiens
  Residues 291-299 of the TADG-16 protein
SQPDPSWPL

SEQ ID NO: 155 - protein, 9 aa, Homo sapiens
  Residues 191-199 of the TADG-16 protein
LQEVQVAII

SEQ ID NO: 156 - protein, 9 aa, Homo sapiens
  Residues 157-165 of the TADG-16 protein
CLQASTFEF

SEQ ID NO: 157 - protein, 9 aa, Homo sapiens
  Residues 122-130 of the TADG-16 protein
SNIYLSPRY

SEQ ID NO: 158 - protein, 9 aa, Homo sapiens
  Residues 182-190 of the TADG-16 protein
DEALPSPHT

SEQ ID NO: 159 - protein, 9 aa, Homo sapiens
  Residues 45-53 of the TADG-16 protein
GEDAELGRW

SEQ ID NO: 160 - protein, 9 aa, Homo sapiens
  Residues 136-144 of the TADG-16 protein
YDIALVKLS

SEQ ID NO: 161 - protein, 9 aa, Homo sapiens
  Residues 170-178 of the TADG-16 protein
DCWVTGWGY

SEQ ID NO: 162 - protein, 9 aa, Homo sapiens
  Residues 243-251 of the TADG-16 protein
ACNKNGLWY

SEQ ID NO: 163 - protein, 9 aa, Homo sapiens
  Residues 163-171 of the TADG-16 protein
FEFENRTDC

SEQ ID NO: 164 - protein, 9 aa, Homo sapiens
  Residues 88-96 of the TADG-16 protein
SDLSDPSGW

SEQ ID NO: 165 - protein, 9 aa, Homo sapiens
  Residues 79-87 of the TADG-16 protein
TAAHCFETY

SEQ ID NO: 166 - protein, 9 aa, Homo sapiens
  Residues 278-296 of the TADG-16 protein
FEWIQKLMA

SEQ ID NO: 167 - protein, 9 aa, Homo sapiens
  Residues 192-200 of the TADG-16 protein
QEVQVAIIN

SEQ ID NO: 168 - protein, 9 aa, Homo sapiens
  Residues 92-100 of the TADG-16 protein
DPSGWMVQF

SEQ ID NO: 169 - protein, 9 aa, Homo sapiens
  Residues 294-302 of the TADG-16 protein
DPSWPLLFF

SEQ ID NO: 170 - protein, 9 aa, Homo sapiens
  Residues 203-211 of the TADG-16 protein
MCNHLFLKY

SEQ ID NO: 171 - protein, 9 aa, Homo sapiens
  Residues 76-84 of the TADG-16 protein
WALTAAHCF

SEQ ID NO: 172 - protein, 9 aa, Homo sapiens
  Residues 165-173 of the TADG-16 protein
FENRTDCWV

SEQ ID NO: 173 - protein, 9 aa, Homo sapiens
  Residues 215-223 of the TADG-16 protein
KDIFGDMVC

SEQ ID NO: 174 - protein, 9 aa, Homo sapiens
  Residues 48-56 of the TADG-16 protein
AELGRWPWQ

SEQ ID NO: 175 - protein, 9 aa, Homo sapiens
  Residues 272-280 of the TADG-16 protein
TNISHHFEW

SEQ ID NO: 176 - protein, 9 aa, Homo sapiens
  Residues 227-235 of the TADG-16 protein
AQGGKDACF

SEQ ID NO: 177 - DNA, 498 bp, Unknown
  WISH cDNA sequence of TADG-16 catalytic domain
tgggcactcacggcggcgcactgctttgaaacgtatagtgaccttagtgatccctccgggtggatggtccagtttggccagctgacttccatgccatccttctggagcctgcaggcctagtacacccgttacttcgtatcgaatatctatctgagccctcgctacctggggaattcaccctatgacattgccttggtgaagctgtctgcacctgtcacctacactaaacacatccagcccatctgtctccaggcctccacatttgagtttgagaaccggacagactgctgggtgactggctgggggtacatcaaagaggatgaggcactgccatctccccacaccctccaggaagttcaggtcgccatcataaacaactctatgtgcaaccacctcttcctcaagtacagtttccgcaaggacatctttggagacatggtttgtgctggcaatgcccaaggcgggaaggatgcctgcttcggtgactcaggtggaccc

SEQ ID NO: 178 - protein, 166 aa, Unknown
  amino acid sequence of TADG-16 catalytic domain
WALTAAHCFETYSDLSDPSGWMVQFGQLTSMPSFWSLQAYYTRYFVSNIYLSPRYLGNSPYDIALVKSLAPVTYTKHIQPICLQASTFEFENRTDCWVTGWGYIKEDEALPSPHTLQEVQVAIINNSMCNHLFLKYSFRKDIFGDMVCAGNAQGGKDACFGDSGGP

SEQ ID NO: 179 - DNA, 500 bp, Unknown
  Accession No. AA620757
ttttttttttttgaagaatgccctgcaaggcatcaactggaatgtgtttattaccaaacaagacagaagagaaccagggcctgacttggcagtggcccaggctgcatgggctcaggtaggctcagaccggccccaggagtgggagagcccagagaagagggaaaaagagtagtggccaggaggggtctggctgggacatgccactctgggccatcagcttctggatccactcaaagtggtggctcatattggtgtagacaccgggccgattgggcgaccacagcccactccccagctcacgactccaatctgataccacagtccattcttgttacaggccaagggtccacctgagtcaccgaagcaggcatccttcccgcacttgggcattgccagcacaaaccatgtctccaaagatgtccttgcggaaactgtacttgaggaagaggtggttgcacatagagttgtttatgatggcgaactgaacttcctggagggtg

(19) United States
(12) Patent Application Publication
(10) Pub. No.: US 2003/0027144 A1
(43) Pub. Date: Feb. 6, 2003

(54) Tumor antigen-derived gene 16 (TADG-16): a novel extracellular serine protease and uses thereof

(75) Inventors: Lowell J. Underwood, Little Rock, AR (US); Timothy J. O'Brien, Little Rock, AR (US); Kazushi Shigemasa, Hiroshima (JP)

Correspondence Address: Dr. Benjamin Adler, Adler & Associates, 8011 Candle Lane, Houston, TX, 77071, US

(73) Assignee: The University of Arkansas for Medical Sciences

(21) Appl. No.: 09/907,187

(22) Filed: Jul. 17, 2001

Related U.S. Application Data
(62) Division of application No. 09/418,527, filed on Oct. 14, 1999, now abandoned.

(51) Int. Cl. C12Q 1/68; G01N 33/574; C07H 21/04; C12P 21/02; C12N 5/06

(52) U.S. Cl. 435/6; 435/7.23; 435/69.1; 435/325; 435/320.1; 530/350; 536/23.5

(57) ABSTRACT

The present invention provides a DNA encoding a TADG-16 protein selected from the group consisting of: (a) isolated DNA which encodes a TADG-16 protein; (b) isolated DNA which hybridizes to isolated DNA of (a) above and which encodes a TADG-16 protein; and (c) isolated DNA differing from the isolated DNAs of (a) and (b) above in codon sequence due to the degeneracy of the genetic code, and which encodes a TADG-16 protein. Also provided is a vector capable of expressing the DNA of the present invention adapted for expression in a recombinant cell and regulatory elements necessary for expression of the DNA in the cell.

### BACKGROUND OF THE INVENTION

1. Field of the Invention

The present invention relates generally to the fields of cellular biology and the diagnosis of neoplastic disease. More specifically, the present invention relates to an extracellular serine protease termed Tumor Antigen Derived Gene-16 (TADG-16), which is expressed in normal ovaries and testes, as well as certain ovarian carcinomas.

2. Description of the Related Art

To date, ovarian cancer remains the number one killer of women with gynecologic malignant hyperplasia. Approximately 75% of women diagnosed with such cancers are already at the high-stage (III and IV) of the disease at their initial diagnosis. During the past 20 years, neither diagnosis nor five year survival have greatly improved for these patients. This is substantially due to the significant number of high-stage initial detections of the disease. Therefore, the challenge remains to develop new markers to improve early diagnosis, and thereby reduce the percentage of high-stage initial diagnoses.

A good tumor marker useful as an indicator of early disease is needed. Extra-cellular proteases have already been implicated in the growth, spread and metastatic progression of many cancers, thereby implying that some extracellular proteases may be candidates for marker of neoplastic development. This is in part due to the ability of malignant cells not only to grow in situ, but to dissociate from the primary tumor and to invade new surfaces (metastasize). The ability to disengage from one tissue and re-engage the surface of another tissue is what results in the morbidity and mortality associated with this disease.

In order for malignant cells to grow, spread or metastasize, they must have the capacity to invade local host tissue, dissociate or shed from the primary tumor, and for metastasis to occur, enter and survive in the bloodstream, implant by invasion into the surface of the target organ and establish an environment conducive for new colony growth (including the induction of angiogenic and growth factors). During this progression, natural tissue barriers have to be degraded, including basement membranes and connective tissue. These barriers further include collagen, laminin, proteoglycans and extracellular matrix glycoproteins, such as fibronectin.

Degradation of these natural barriers, both surrounding the primary tumor and at sites of metastatic invasion, is believed to be brought about by the action of extracellular proteases. Proteases have been classified into four families: serine proteases, metallo-proteases, aspartic proteases and cysteine proteases. Many proteases have been shown to be involved in the human disease process and these enzymes are targets for inhibition by new therapeutic agents.

Certain individual proteases have already been shown to be induced and overexpressed in a diverse group of cancers, and as such, are potential candidates for markers useful for early diagnosis and possibly therapeutic intervention. Examples of proteases, encompassing members of the metallo-proteases, serine proteases, and cysteine proteases, are listed below.

TABLE 1
Protease Expression in Various Cancers

| | Gastric | Brain | Breast | Ovarian |
|---|---|---|---|---|
| Serine | uPA | uPA | NES-1 | NES-1 |
| Proteases | PAI-1 | PAI-1 | uPA | uPA |
| | | tPA | | PAI-2 |
| Cysteine | Cathepsin B | Cathepsin L | Cathepsin B | Cathepsin B |
| Proteases | Cathepsin L | | Cathepsin L | Cathepsin L |
| Metallo- | Matrilysin* | Matrilysin | Stromelysin-3 | MMP-2 |
| proteases | Collagenase* | Stromelysin | MMP-8 | |
| | Stromelysin-1* | Gelatinase B | MMP-9 | |
| | | | Gelatinase A | |

uPA, Urokinase-type plasminogen activator; tPA, Tissue-type plasminogen activator; PAI-I, Plasminogen activator 0 inhibitors; PAI-2, Plasminogen activator inhibitors; NES-1, Normal epithelial cell-specific-1; MMP, Matrix P metallo-protease. *Overexpressed in gastrointestinal ulcers.

Significantly, there is a good body of evidence supporting the down regulation or inhibition of individual proteases and a subsequent reduction in invasive capacity or malignancy. In work by Clark et al., (Peptides, 14, 1021-8 (1993)) inhibition of in vitro growth of human small cell lung cancer was demonstrated using a general serine protease inhibitor. More recently, Torres-Rosedo et al., (Proc. Natl. Acad. Sci. USA, 90, 7181-7185 (1993)) demonstrated an inhibition of hepatoma tumor cell growth using specific antisense inhibitors for the serine protease hepsin gene. Metastatic potential has also been shown to be reduced using a synthetic inhibitor (batimastat) of metallo-protease in a mouse model with melanoma cells. Powell et al. (Cancer Research, 53, 417-422 (1993)) presented evidence to confirm that the expression of extracellular proteases in relatively non-invasive tumor cells enhances their malignant progression using a tumor-genic, but non-metastatic, prostate cell line. Specifically, Powell et al. demonstrated enhanced metastasis after introducing and expressing the PUMP-1 metallo-protease gene. There is also a body of data to support the notion that expression of cell surface proteases on relatively non-metastatic cell types increases the invasive potential of such cells.

Extracellular proteases have been directly associated with tumor growth, shedding of tumor cells and invasion of target organs by tumors. Individual classes of proteases are involved in, but not limited to, (a) digestion of stroma surrounding the initial tumor area; (b) digestion of the cellular adhesion molecules to allow dissociation of tumor cells; and (c) invasion of the basement membrane for metastatic growth and the activation of both tumor growth factors and angiogenic factors.

Interfering in the intracellular signal transduction pathways provides mechanisms for numerous therapeutic applications. While several proteins have been identified that interfere with various signal transduction mechanisms, novel proteins involved in signal transduction pathways are important to provide alternatives for therapy and drug development.

The prior art is deficient in that the prior art lacks the nucleotide and amino acid sequences corresponding to tumor antigen-derived gene 16 (TADG-16). The prior art further lacks effective means of screening to identify proteases, specifically TADG-16, expressed in normal ovaries and testes and certain ovarian carcinomas. The present invention fulfills this longstanding need and desire in the art.

### SUMMARY OF THE INVENTION

This invention describes a new serine protease enzyme. The TADG-16 enzyme contains the characteristic features of a serine protease, including the conserved catalytic triad (His-Asp-Ser) and a secretion signal sequence. The TADG-16 transcript is present in carcinomas and normal ovarian tissues as well as in normal testes. Because TADG-16 is secreted and has a potential for extracellular activation, TADG-16 may have a role in normal or aberrant physiological activity of ovary or testes.

In one embodiment of the present invention, there is provided a DNA encoding a tumor antigen-derived gene (TADG-16) protein, selected from the following: (a) an isolated DNA which encodes a TADG-16 protein; (b) an isolated DNA which hybridizes under high stringency conditions to the isolated DNA of (a) above and which encodes a TADG-16 protein; and (c) an isolated DNA differing from the isolated DNAs of (a) and (b) above in codon sequence due to the degeneracy of the genetic code, and which encodes a TADG-16 protein. The embodiment further includes a vector comprising the TADG-16 DNA and regulatory elements necessary for expression of the DNA in a cell. Additionally embodied is a vector in which the TADG-16 DNA is positioned in reverse orientation relative to the regulatory elements such that TADG-16 antisense mRNA is produced.

In another embodiment of the present invention, there is provided an isolated and purified TADG-16 protein coded for by DNA selected from the following: (a) an isolated DNA which encodes a TADG-16 protein; (b) an isolated DNA which hybridizes under high stringency conditions to isolated DNA of (a) above and which encodes a TADG-16 protein; and (c) an isolated DNA differing from the isolated DNAs of (a) and (b) above in codon sequence due to the degeneracy of the genetic code, and which encodes a TADG-16 protein.

In yet another embodiment of the present invention, there is provided a method for detecting TADG-16 mRNA in a sample, comprising the steps of (a) contacting a sample with a probe which is specific for TADG-16; and (b) detecting binding of the probe to TADG-16 mRNA in the sample. In still yet another embodiment of the present invention, there is provided a kit for detecting TADG-16 mRNA, comprising an oligonucleotide probe specific for TADG-16. A label for detection is further embodied in the kit.

The present invention additionally embodies a method of detecting TADG-16 protein in a sample, comprising the steps of (a) contacting a sample with an antibody which is specific for TADG-16 or a fragment thereof; and (b) detecting binding of the antibody to TADG-16 protein in the sample. Similarly, the present invention also embodies a kit for detecting TADG-16 protein, comprising an antibody specific for TADG-16 protein or a fragment thereof. Means for detection of the antibody is further embodied in the kit.

In another embodiment, the present invention provides an antibody specific for the TADG-16 protein or a fragment thereof.

In yet another embodiment, the present invention provides a method of screening for compounds that inhibit TADG-16, comprising the steps of (a) contacting a sample comprising TADG-16 protein with a compound; and (b) assaying for TADG-16 protease activity. Typically, a decrease in the TADG-16 protease activity in the presence of the compound relative to TADG-16 protease activity in the absence of the compound is indicative of a compound that inhibits TADG-16.

In still yet another embodiment of the present invention, there is provided a method of inhibiting expression of TADG-16 in a cell, comprising the step of (a) introducing a vector into a cell, whereupon expression of the vector produces TADG-16 antisense mRNA in the cell which hybridizes to endogenous TADG-16 mRNA, thereby inhibiting expression of TADG-16 in the cell.

Further embodied by the present invention, there is provided a method of inhibiting a TADG-16 protein in a cell, comprising the step of (a) introducing an antibody specific for a TADG-16 protein or a fragment thereof into a cell, whereupon binding of the antibody to the TADG-16 protein inhibits the TADG-16 protein.

In another embodiment of the present invention, there is provided a method of targeted therapy to an individual, comprising the step of (a) administering a compound containing a targeting moiety and a therapeutic moiety to an individual, wherein the targeting moiety is specific for TADG-16.

In another embodiment of the present invention, there is provided a method of diagnosing cancer in an individual, comprising the steps of (a) obtaining a biological sample from an individual; and (b) detecting TADG-16 in the sample. Typically, the presence of TADG-16 in the sample is indicative of the presence of carcinoma in the individual and the absence of TADG-16 in the sample is indicative of the absence of carcinoma in the individual.

In another embodiment of the present invention, there is provided a method of vaccinating an individual against TADG-16, comprising the steps of (a) inoculating an individual with a TADG-16 protein or fragment thereof that lacks TADG-16 protease activity. It is intended that inoculation with the TADG-16 protein or fragment thereof elicits an immune response in the individual, thereby vaccinating the individual against TADG-16.

In another embodiment of the present invention, there is provided an immunogenic composition, comprising an immunogenic fragment of TADG-16 and an appropriate adjuvant.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows an alignment of a portion of the TADG-16 protein sequence (SEQ ID No. 7) with other known proteases (Prom, Protease M (SEQ ID No. 3); Tryl, Trypsinogen 1 (SEQ ID No. 4); SCCE, Stratum corneum chymotryptic like enzyme (SEQ ID No. 5); and Heps, Hepsin (SEQ ID No. 6)).

FIG. 2 shows Northern blot analysis of multiple human tissues using the radioactively labeled catalytic domain as a probe. The 1.4 Kb TADG-16 transcript is present in normal human testes and in certain ovarian tumors, but is not detectable a t significant levels in other tissues examined. Hybridization of mRNA to β-tubulin is shown as an internal control.

FIG. 3A shows the nucleotide and predicted amino acid sequence of the original subclone from the WISH cDNA containing the TADG-16 catalytic domain.

FIG. 3B shows a sequence identified from the EST database (Accession #AA620757) with homology to the TADG-16 catalytic domain (encoding bases 614 to 1129) and including the 3′-untranslated region and poly (A) tail of the TADG-16 transcript.

FIG. 4 shows the nucleotide sequence of the TADG-16 cDNA and the predicted amino acid sequence. The cDNA corresponding to TADG-16 contains a Kozak's consensus sequence (boxed nucleotides) for the initiation of translation from which a putative protein of 314 amino acids is encoded. The protein contains a secretion signal sequence (italicized) and the conserved amino acids of the catalytic triad of the serine protease family (circled) in the appropriate context (underlined residues). The cDNA also contains a polyadenylation sequence in the 3′ untranslated region (underlined nucleotides).

FIG. 5 shows TADG-16 (and β-tubulin) expression in normal and carcinoma cell lines.

FIG. 6 shows TADG-16 expression in normal (N), benign (B), low malignant potential (LMP) tumors and carcinomas (C).

FIG. 6A shows quantitative PCR of TADG-16 (250 bp) and internal control, β-tubulin (470 bp). Lanes 1-3, normal ovary (cases 5-7, respectively); Lanes 4-5, benign mucinous adenoma tumor (cases 8 & 11, respectively); Lane 6, serous LMP tumor (case 14); Lanes 7-8, clear cell carcinoma (cases 20 & 21, respectively); Lanes 9-11, serous adenocarcinoma (cases 22, 29 and 32, respectively); Lane 12, endometrioid adenocarcinoma (case 35).

FIG. 6B shows a graph of expression of TADG-16 in normal ovaries and ovarian benign, LMP and carcinoma tumors.

### DETAILED DESCRIPTION OF THE INVENTION

This invention describes a new serine protease enzyme complementary to the series of proteases already identified and characterized in ovarian carcinoma. The TADG-16 enzyme contains the characteristic features of all serine proteases, including the conserved catalytic triad of His-Asp-Ser and a signal secretion sequence. The transcript for this enzyme is present in carcinomas and normal ovarian tissues as well as in normal testes. Because TADG-16 is secreted and has a potential for extracellular activation, TADG-16 may have a role in normal or aberrant physiological activity (i.e., normal or carcinomatous growth) of ovary or testes. Furthermore, because of the presence of TADG-16 mRNA in normal testes, there is a potential role for TADG-16 in normal testicular function (e.g., sterility).

The TADG-16 cDNA is 1129 base pairs long (SEQ ID No. 1) and encodes a 314 amino acid protein (SEQ ID No. 2).

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

As used herein, the term “cDNA” shall refer to the DNA copy of the mRNA transcript of a gene.

As used herein, the term “derived amino acid sequence” shall mean the amino acid sequence determined by reading the triplet sequence of nucleotide bases in the cDNA.

As used herein the term “screening a library” shall refer to the process of using a labeled probe to check whether, under the appropriate conditions, there is a sequence complementary to the probe present in a particular DNA library. In addition, “screening a library” could be performed by PCR.

As used herein, the term “PCR” refers to the Polymerase Chain Reaction that is the subject of U.S. Pat. Nos. 4,683,195 and 4,683,202 to Mullis, as well as other improvements to the process/technique of PCR now known in the art.

The amino acid described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J Biol. Chem., 243:3552-59 (1969), abbreviations for amino acid residues are shown in Table 2.

TABLE 2

Symbol

| 1 Letter | 3 Letter | Amino acid |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. The structure is discussed herein according to the normal convention of giving only the 5′ to 3′ sequence of the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included near the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide”, as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to a n oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementary with the sequence or hybridize therewith and thereby form the template for the synthesis of the extension product.

As used herein, the terms “restriction endonucleases” and “restriction enzymes” refer to enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90% or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

A “heterologous” region of the DNA construct is a n identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. In another example, coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

Proteins can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized calorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

A particular assay system developed and utilized in the art is known as a receptor assay. In a receptor assay, the material to be assayed is appropriately labeled and then certain cellular test colonies are inoculated with a quantitiy of both the label after which binding studies are conducted to determine the extent to which the labeled material binds to the cell receptors. In this way, differences in affinity between materials can be ascertained.

An assay useful in the art is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluminescence is then measured photometrically, and dose response curves are obtained and compared to those of known ligands. The foregoing protocol is described in detail in U.S. Pat. No. 4,981,784.

As used herein, the term “host” is meant to include not only prokaryotes but also eukaryotes such as yeast, plant and animal cells. A recombinant DNA molecule or gene which encodes a human TADG-16 protein of the present invention can be used to transform a host using any of the techniques commonly known to those of ordinary skill in the art. Especially preferred is the use of a vector containing coding sequences for the gene which encodes a human TADG-16 protein of the present invention for purposes of prokaryote transformation. Prokaryotic hosts may include E. coli, S. tymphimurium, Serratia marcescens and Bacillus subtilis. Eukaryotic hosts include yeasts such as Pichia pastoris, mammalian cells and insect cells.

In general, expression vectors containing promoter sequences which facilitate the efficient transcription of the inserted DNA fragment are used in connection with the host. The expression vector typically contains an origin of replication, promoter(s), terminator(s), as well as specific genes which are capable of providing phenotypic selection in transformed cells. The transformed hosts can be fermented and cultured according to means known in the art to achieve optimal cell growth.

The invention includes a substantially pure DNA encoding a TADG-16 protein, a strand of which DNA will hybridize at high stringency to a probe containing a sequence of at least 15 consecutive nucleotides of SEQ ID No. 1. The protein encoded by the DNA of this invention may share at least 80% sequence identity (preferably 85%, more preferably 90%, and most preferably 95%) with the amino acids shown in SEQ ID No. 2. More preferably, the DNA includes the coding sequence of the nucleotides shown in SEQ ID No. 1, or a degenerate variant of such a sequence.

The probe to which the DNA of the invention hybridizes preferably consists of a sequence of at least 20 consecutive nucleotides, more preferably 40 nucleotides, even more preferably 50 nucleotides, and most preferably 100 nucleotides or more (up to 100%) of the coding sequence of the nucleotides shown in SEQ ID No. 1 or the complement thereof. Such a probe is useful for detecting expression of TADG-16 in a cell by a method including the steps of (a) contacting mRNA obtained from the cell with the labeled hybridization probe; and (b) detecting hybridization of the probe with the mRNA.

This invention also includes a substantially pure DNA containing a sequence of at least 15 consecutive nucleotides (preferably 20, more preferably 30, even more preferably 50, and most preferably all) of the region from nucleotides 1 to 3147 of the nucleotides shown in SEQ ID No. 1.

By “high stringency” is meant DNA hybridization and wash conditions characterized by high temperature and low salt concentration, e.g., wash conditions of 65° C. at a salt concentration of approximately 0.1× SSC, or the functional equivalent thereof. For example, high stringency conditions may include hybridization a t about 42° C. in the presence of about 50% formamide; a first wash a t about 65° C. with about 2× SSC containing 1% SDS; followed by a second wash at about 65° C. with about 0.1× SSC.

By “substantially pure DNA” is meant DNA that is not part of a milieu in which the DNA naturally occurs, by virtue of separation (partial or total purification) of some or all of the molecules of that milieu, or by virtue of alteration of sequences that flank the claimed DNA. The term therefore includes, for example, a recombinant DNA which is incorporated into a vector, into an autonomously replicating plasmid or virus, or into the genomic DNA of a prokaryote or eukaryote; or which exists as a separate molecule (e.g., a cDNA or a genomic or cDNA fragment produced by polymerase chain reaction (PCR) or restriction endonuclease digestion) independent of other sequences. It also includes a recombinant DNA which is part of a hybrid gene encoding additional polypeptide sequence, e.g., a fusion protein. Also included is a recombinant DNA which includes a portion of the nucleotides shown in SEQ ID No. 1 which encodes an alternative splice variant of TADG-16.

The DNA may have at least about 70% sequence identity to the coding sequence of the nucleotides shown in SEQ ID No. 1, preferably at least 75% (e.g., at least 80%); and most preferably at least 90%. The identity between two sequences is a direct function of the number of matching or identical positions. When a subunit position in both of the two sequences is occupied by the same monomeric subunit, e.g., if a given position is occupied by an adenine in each of two DNA molecules, then they are identical at that position. For example, if 7 positions in a sequence 10 nucleotides in length are identical to the corresponding positions in a second 10-nucleotide sequence, then the two sequences have 70% sequence identity. The length of comparison sequences will generally be at least 50 nucleotides, preferably at least 60 nucleotides, more preferably at least 75 nucleotides, and most preferably 100 nucleotides. Sequence identity is typically measured using sequence analysis software (e.g., Sequence Analysis Software Package of the Genetics Computer Group, University of Wisconsin Biotechnology Center, 1710 University Avenue, Madison, Wis. 53705).

The present invention is directed towards a vector comprising a DNA sequence which encodes a TADG-16 protein, wherein the vector is capable of replication in a host cell, wherein the vector comprises, in operable linkage: a) an origin of replication; b) a promoter; and c) a DNA sequence coding for the TADG-16 protein. Preferably, the vector of the present invention contains a portion of the DNA sequence shown in SEQ ID No. 1.

A “vector” may be defined as a replicable nucleic acid construct, e.g., a plasmid or viral nucleic acid. Vectors may be used to amplify and/or express nucleic acid encoding TADG-16 protein. An expression vector is a replicable construct in which a nucleic acid sequence encoding a polypeptide is operably linked to suitable control sequences capable of effecting expression of the polypeptide in a cell. The need for such control sequences will vary depending upon the cell selected and the transformation method chosen. Generally, control sequences include a transcriptional promoter and/or enhancer, suitable mRNA ribosomal binding sites, and sequences which control the termination of transcription and translation.

Methods which are well known to those skilled in the art can be used to construct expression vectors containing appropriate transcriptional and translational control signals. See for example, the techniques described in Sambrook et al., 1989, Molecular Cloning: A Laboratory Manual (2nd Ed.), Cold Spring Harbor Press, N.Y. A gene and its transcription control sequences are defined as being “operably linked” if the transcription control sequences effectively control the transcription of the gene. Vectors of the invention include, but are not limited to, plasmid vectors and viral vectors. Preferred viral vectors of the invention are those derived from retroviruses, adenovirus, adeno-associated virus, SV40 virus, or herpes viruses.

By a “substantially pure protein” is meant a protein which has been separated from at least some of those components which naturally accompany it. Typically, the protein is substantially pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially pure TADG-16 protein may be obtained, for example, by extraction from a natural source; by expression of a recombinant nucleic acid encoding an TADG-16 polypeptide; or by chemically synthesizing the protein. Purity can be measured by any appropriate method, e.g., column chromatography such as immunoaffinity chromatography using an antibody specific for TADG-16, polyacrylamide gel electrophoresis, or HPLC analysis. A protein is substantially free of naturally associated components when it is separated from at least some of those contaminants which accompany it in its natural state. Thus, a protein which is chemically synthesized or produced in a cellular system different from the cell from which it naturally originates will be, by definition, substantially free from its naturally associated components. Accordingly, substantially pure proteins include eukaryotic proteins synthesized in E. coli, other prokaryotes, or any other organism in which they do not naturally occur.

In addition to substantially full-length proteins, the invention also includes fragments (e.g., antigenic fragments) of the TADG-16 protein (SEQ ID No. 2). As used herein, “fragment,” as applied to a polypeptide, will ordinarily be at least 10 residues, more typically at least 20 residues, and preferably at least 30 (e.g., 50) residues in length, but less than the entire, intact sequence. Fragments of the TADG-16 protein can be generated by methods known to those skilled in the art, e.g., by enzymatic digestion of naturally occurring or recombinant TADG-16 protein, by recombinant DNA techniques using an expression vector that encodes a defined fragment of TADG-16, or by chemical synthesis. The ability of a candidate fragment to exhibit a characteristic of TADG-16 (e.g., binding to an antibody specific for TADG-16) can be assessed by methods described herein. Purified TADG-16 or antigenic fragments of TADG-16 can be used to generate new antibodies or to test existing antibodies (e.g., as positive controls in a diagnostic assay) by employing standard protocols known to those skilled in the art.

Included in this invention are polyclonal antisera generated by using TADG-16 or a fragment of TADG-16 as the immunogen in, e.g., rabbits. Standard protocols for monoclonal and polyclonal antibody production known to those skilled in this art are employed. The monoclonal antibodies generated by this procedure can be screened for the ability to identify recombinant TADG-16 cDNA clones, and to distinguish them from known cDNA clones.

Further included in this invention are TADG-16 proteins which are encoded at least in part by portions of SEQ ID No. 2, e.g., products of alternative mRNA splicing or alternative protein processing events, or in which a section of TADG-16 sequence has been deleted. The fragment, or the intact TADG-16 polypeptide, may be covalently linked to another polypeptide, e.g., which acts as a label, a ligand or a means to increase antigenicity.

The invention also includes a polyclonal or monoclonal antibody which specifically binds to TADG-16. The invention encompasses not only an intact monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin.

In one embodiment, the antibody, or a fragment thereof, may be linked to a toxin or to a detectable label, e.g., a radioactive label, non-radioactive isotopic label, fluorescent label, chemiluminescent label, paramagnetic label, enzyme label, or colorimetric label. Examples of suitable toxins include diphtheria toxin, Pseudomonas exotoxin A, ricin, and cholera toxin. Examples of suitable enzyme labels include malate hydrogenase, staphylococcal nuclease, delta-5-steroid isomerase, alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, acetylcholinesterase, etc. Examples of suitable radioisotopic labels include ³H, ¹²⁵I, ¹³¹I, ³²P, ³⁵ S, ¹⁴C.

Paramagnetic isotopes for purposes of in vivo diagnosis can also be used according to the methods of this invention. There are numerous examples of elements that are useful in magnetic resonance imaging. For discussions on in vivo nuclear magnetic resonance imaging, see, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415. Examples of suitable fluorescent labels include a fluorescein label, an isothiocyalate label, a rhodamine label, a phycoerythrin label, a phycocyanin label, a n allophycocyanin label, an ophthaldehyde label, a fluorescamine label, etc. Examples of chemiluminescent labels include a luminal label, an isoluminal label, an aromatic acridinium ester label, a n imidazole label, an acridinium salt label, an oxalate ester label, a luciferin label, a luciferase label, an aequorin label, etc.

Those of ordinary skill in the art will know of other suitable labels which may be employed in accordance with the present invention. The binding of these labels to antibodies or fragments thereof can be accomplished using standard techniques commonly known to those of ordinary skill in the art. Typical techniques are described by Kennedy et al., (1976) Clin. Chim. Acta 70, 1-31; and Schurs et al., (1977) Clin. Chim. Acta .81, 1-40. Coupling techniques mentioned in the latter are the glutaraldehyde method, the periodate method, the dimaleimide method, the m-maleimidobenzyl-N-hydroxy-succinimide ester method. All of these methods are incorporated by reference herein.

Also within the invention is a method of detecting TADG-16 protein in a biological sample, which includes the steps of contacting the sample with the labeled antibody, e.g., radioactively tagged antibody specific for TADG-16, and determining whether the antibody binds to a component of the sample.

As described herein, the invention provides a number of diagnostic advantages and uses. For example, the TADG-16 protein is useful in diagnosing cancer in different tissues since this protein is highly overexpressed in tumor cells. Antibodies (or antigen-binding fragments thereof) which bind to an epitope specific for TADG-16, are useful in a method of detecting TADC-16 protein in a biological sample for diagnosis of cancerous or neoplastic transformation. This method includes the steps of obtaining a biological sample (e.g., cells, blood, plasma, tissue, etc.) from a patient suspected of having cancer, contacting the sample with a labeled antibody (e.g., radioactively tagged antibody) specific for TADG-16, and detecting the TADG-16 protein using standard immunoassay techniques such as an ELISA. Antibody binding to the biological sample indicates that the sample contains a component which specifically binds to an epitope within TADG-16.

Likewise, a standard Northern blot assay can be used to ascertain the relative amounts of TADG-16 mRNA in a cell or tissue obtained from a patient suspected of having cancer, in accordance with conventional Northern hybridization techniques known to those of ordinary skill in the art. This Northern assay uses a hybridization probe, e.g., radiolabelled TADG-16 cDNA, either containing the full-length, single stranded DNA having a sequence complementary to SEQ ID No. 1, or a fragment of that DNA sequence at least 20 (preferably at least 30, more preferably at least 50, and most preferably at least 100) consecutive nucleotides in length. The DNA hybridization probe can be labeled by any of the many different methods known to those skilled in this art.

Antibodies to the TADG-16 protein can be used in an immunoassay to detect increased levels of TADG-16 protein expression in tissues suspected of neoplastic transformation. These same uses can be achieved with Northern blot assays and analyses.

The TADG-16 cDNA is 1129 base pairs long (SEQ ID No. 1) encoding for a 314 amino acid protein (SEQ ID No. 2). The availability of the TADG-16 gene provides numerous utilities. For example, the TADG-16 gene can be used as a diagnostic or therapeutic target in ovarian and other carcinomas, including breast, prostate, lung and colon.

The present invention is directed to DNA encoding a tumor antigen-derived gene (TADG-16) protein, selected from (a) an isolated DNA which encodes a TADG-16 protein; (b) an isolated DNA which hybridizes under high stringency conditions to the isolated DNA of (a) above and which encodes a TADG-16 protein; and (c) an isolated DNA differing from the isolated DNAs of (a) and (b) above in codon sequence due to the degeneracy of the genetic code, and which encodes a TADG-16 protein. It is preferred that the DNA has the sequence shown in SEQ ID No. 1 and the TADG-16 protein has the amino acid sequence shown in SEQ ID No. 2.

The present invention is directed toward a vector comprising the TADG-16 DNA and regulatory elements necessary for expression of the DNA in a cell, or a vector in which the TADG-16 DNA is positioned in reverse orientation relative to the regulatory elements such that TADG-16 antisense mRNA is produced. An antisense molecule corresponding to TADG-16 mRNA is shown in SEQ ID No. 16. The invention is also directed toward host cells transfected with either of the above-described vector(s). Representative host cells are bacterial cells, mammalian cells, plant cells and insect cells. Preferably, the bacterial cell is E. coli.

The present invention is directed toward an isolated and purified TADG-16 protein coded for by DNA selected from the following: (a) an isolated DNA which encodes a TADG-16 protein; (b) an isolated DNA which hybridizes under high stringency conditions to isolated DNA of (a) above and which encodes a TADG-16 protein; and (c) an isolated DNA differing from the isolated DNAs of (a) and (b) above in codon sequence due to the degeneracy of the genetic code, and which encodes a TADG-16 protein. Preferably, the protein has the amino acid sequence shown in SEQ ID No. 2.

The present invention is directed toward a method for detecting TADG-16 mRNA in a sample, comprising the steps of (a) contacting a sample with a probe which is specific for TADG-16; and (b) detecting binding of the probe to TADG-16 mRNA in the sample. The present invention is also directed toward a method of detecting TADG-16 protein in a sample, comprising the steps of (a) contacting a sample with an antibody which is specific for TADG-16 or a fragment thereof; and (b) detecting binding of the antibody to TADG-16 protein in the sample. Generally, the sample is a biological sample; preferably, the biological sample is from an individual; and typically, the individual is suspected of having cancer.

The present invention is directed toward a kit for detecting TADG-16 mRNA, comprising an oligonucleotide probe, wherein the probe is specific for TADG-16. The kit may further comprise a label with which to label the probe; and means for detecting the label. The present invention is additionally directed toward a kit for detecting TADG-16 protein, comprising an antibody which is specific for TADG-16 protein or a fragment thereof. The kit may further comprise means to detect the antibody.

The present invention is directed toward a antibody which is specific for TADG-16 protein or a fragment thereof.

The present invention is directed toward a method of screening for compounds that inhibit TADG-16, comprising the steps of: (a) contacting a sample containing TADG-16 protein with a compound; and (b) assaying for TADG-16 protease activity. Typically, a decrease in the TADG-16 protease activity in the presence of the compound relative to TADG-16 protease activity in the absence of the compound is indicative of a compound that inhibits TADG-16.

The present invention is directed toward a method of inhibiting expression of TADG-16 in a cell, comprising the step of: (a) introducing a vector expressing TADG-16 antisense mRNA into a cell which hybridizes to endogenous TADG-16 mRNA, thereby inhibiting expression of TADG-16 in the cell. Generally, the inhibition of TADG-16 expression is for treating cancer.

The present invention is directed toward a method of inhibiting a TADG-16 protein in a cell, comprising the step of (a) introducing an antibody specific for a TADG-16 protein or a fragment thereof into a cell which inhibits the TADG-16 protein. Generally, the inhibition of the TADG-16 protein is for treating cancer.

The present invention is directed toward a method of targeted therapy to an individual, comprising the step of (a) administering a compound having a targeting moiety and a therapeutic moiety to an individual, wherein the targeting moiety is specific for TADG-16. Representative targeting moiety are a n antibody specific for TADG-16, a ligand that binds TADG-16 or a ligand binding domain of TADG-16, e.g., a CUB domain, an LDLR domain, etc. Likewise, a representative therapeutic moiety is a radioisotope, a toxin, a chemotherapeutic agent, an immune stimulant or a cytotoxic agent. Typically, the above-described method is useful when the individual suffers from ovarian cancer, breast cancer, lung cancer, prostate cancer, colon cancer or other cancers in which TADG-16 is overexpressed.

The present invention is directed toward a method of diagnosing cancer in an individual, comprising the steps of (a) obtaining a biological sample from an individual; and (b) detecting TADG-16 in the sample. Generally, the presence of TADG-16 in the sample is indicative of the presence of carcinoma in the individual, and the absence of TADG-16 in the sample is indicative of the absence of carcinoma in the individual. Typically, the biological sample is blood, urine, saliva tears, interstitial fluid, ascites fliud, tumor tissue biopsy or circulating tumor cells. Representative means of detecting TADG-16 are by Northern blot, Western blot, PCR, dot blot, ELIZA sandwich assay, radioimmunoassay, DNA array chips or flow cytometry (after labeling tumor cells). This method may be useful in diagnosing cancers such as ovarian, breast, lung, colon, prostate and others with increased TADG-16 expression.

The present invention is also directed to an antisense oligonucleotide having the nucleotide sequence complementary to a TADG-16 mRNA sequence. The present invention is also directed to a composition comprising such an antisense oligonucleotide and a physiologically acceptable carrier therefore.

The present invention is also directed to a method of treating a neoplastic state in an individual in need of such treatment, comprising the step of administering to said individual an effective dose of an antisense oligonucleotide. Preferably, the neoplastic state is ovarian cancer, breast cancer and other cancers that exhibit TADG-16 overexpression. For such therapy, the oligonucleotides alone or in combination with other anti-neoplastic agents can be formulated for a variety of modes of administration, including systemic, topical or localized administration. Techniques and formulations generally can be found in Remington's Pharmaceutical Sciences (Mack Publishing Co., Easton, Pa.). The oligonucleotide active ingredient is generally combined with a pharamceutically accceptable carrier such as a diluent or excipient which can include fillers, extenders, binders, wetting agents, disintergrants, surface active agents or lubricants, depending on the nature of the mode of administration and dosage forms. Typical dosage forms include tablets, powders, liquid preparations including suspensions, emulsions, and solutions, granules, capsules and suppositories, as well as liquid preparations for injections, including liposome preparations.

For systemic administration, injection is preferred, including intramuscular, intravenous, intraperitoneal and subcutaneous. For injection, the oligonucleotides of the invention are formulated in liquid solutions, preferably in physiologically compatible buffers. In addition, the oligonucleotides can be formulated in solid form and redissolved or suspended immediately prior to use. Lyophilized forms are also incldued. Dosages that can be used for systemic administration preferably range from about 0.01 mg/kg to 50 mg/kg administered once or twice per day. However, different dosing schedules can be utilized depending on (1) the potency of an individual oligonucleotide at inhibiting the activity of its target DNA, (2) the severity or extent of the pathological disease state, or (3) the pharmacokinetic behavior of a given oligonucleotide.

The present invention is directed toward a method of vaccinating an individual against TADG-16, comprising the steps of (a) inoculating an individual with a TADG-16 protein or fragment thereof which lacks TADG-16 protease activity. The inoculation with the TADG-16 protein or fragment thereof elicits an immune response in the individual, thereby vaccinating the individual against TADG-16. The vaccination with TADG-16 described herein is intended for an individual who has cancer, is suspected of having cancer or is a t risk of getting cancer. The present invention is also directed toward an immunogenic composition, comprising an immunogenic fragment of TADG-16 and an appropriate adjuvant. Generally, the TADG-16 fragment useful for vaccinating an individual consists of a 9-residue fragment up to and including a 20-residue fragment. Preferably, the 9-residue fragments have a sequence such as SEQ ID Nos. 17, 18, 19, 77, 78, 79, 80, 97, 98, 99, 137, 138, 139, 140 or 141. Other TADG-16 fragment useful for vaccinating an individual may be readily determined by an individual having ordinary skill in this art using routine techniques.

The present invention is further directed to a method of regulating the expression of the TADG-16 protein by designing antisense oligonucleotides directed to the DNA encoding the TADG -16 protein. A person having ordinary skill in this art would be able design such antisense oligonucleotides without undue experimentation.

The following examples are given for the purpose of illustrating various embodiments of the invention and are not meant to limit the present invention in any fashion.

### EXAMPLE 1

Cloning of the TADG-16 Catalytic Domain

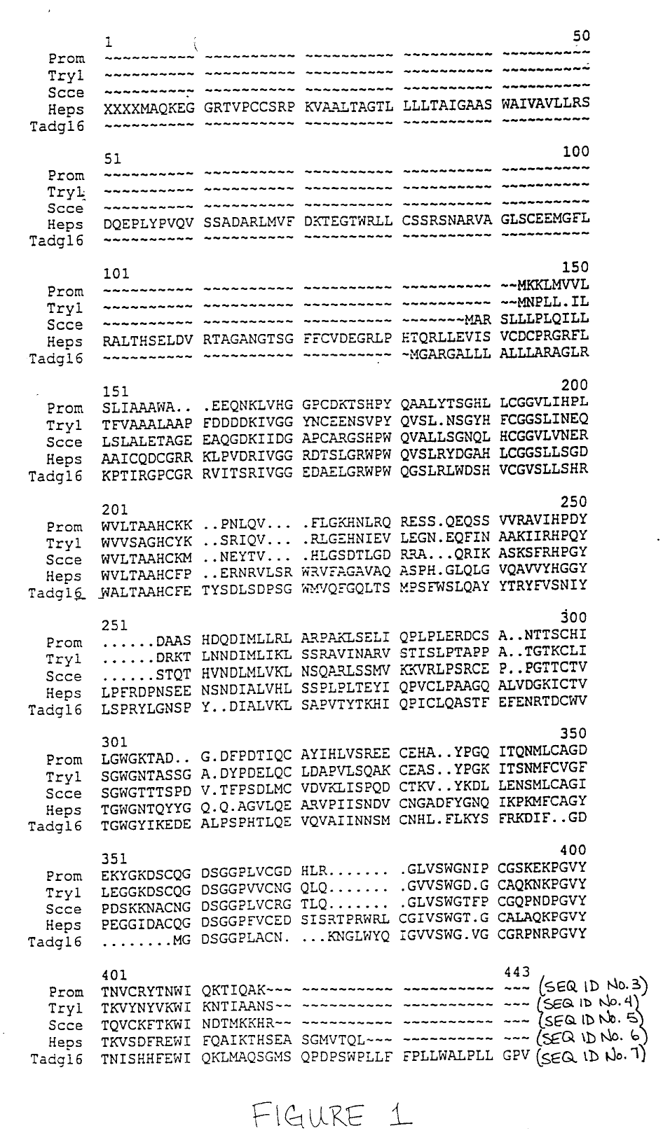

Using WISH (an amnion derived cell line) cDNA (ATCC) as a template for PCR with degenerate primers designed to the conserved regions surrounding the invariant histidine and serine residues of the catalytic triad of the serine protease family of proteins, a 498 base pair product was obtained that was similar in particular consensus sequences to other known serine proteases (FIG. 1).

The sequences of the degenerate primers used in the initial PCR are as follows:

| | | |
|---|---|---|
| Serp-S (Sense): | 5′-TGGGTIGTIACIGCIGGICA(CT)TG-3′ | (SEQ ID No:8); and |
| Serp-S (Antisense): | 5′-A(AG)IGGICCICCI(CG)(TA)(AG)TCICC-3′ | (SEQ ID No:9). |

Reactions were carried out as described by Underwood et al. (Cancer Res., 59, 4435-9 (1999)).

### EXAMPLE 2

Detection of TADG-16 mRNA

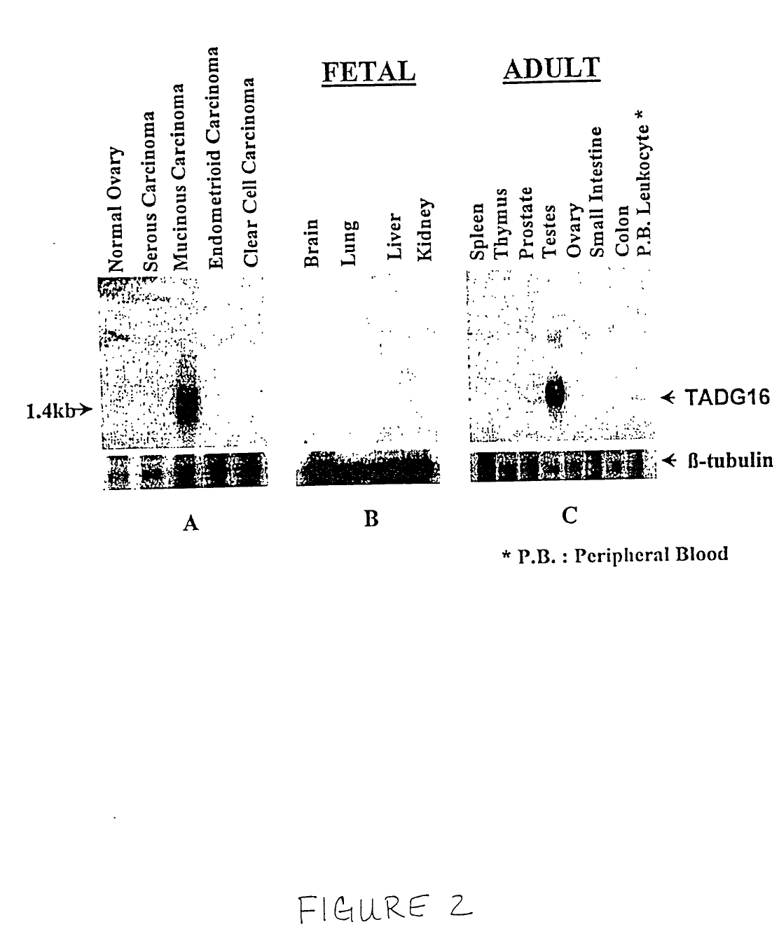

Using the radioactively labeled catalytic domain as a probe, Northern blot analysis of multiple human tissues revealed that TADG-16 is highly expressed in normal human testes and in some ovarian tumors, but not detectable at significant levels in other tissues examined (FIG. 2). More importantly, Northern analysis showed that the TADG-16 transcript is approximately 1.4 kilobases in length.

### EXAMPLE 3

Sequence Analysis of TADG-16

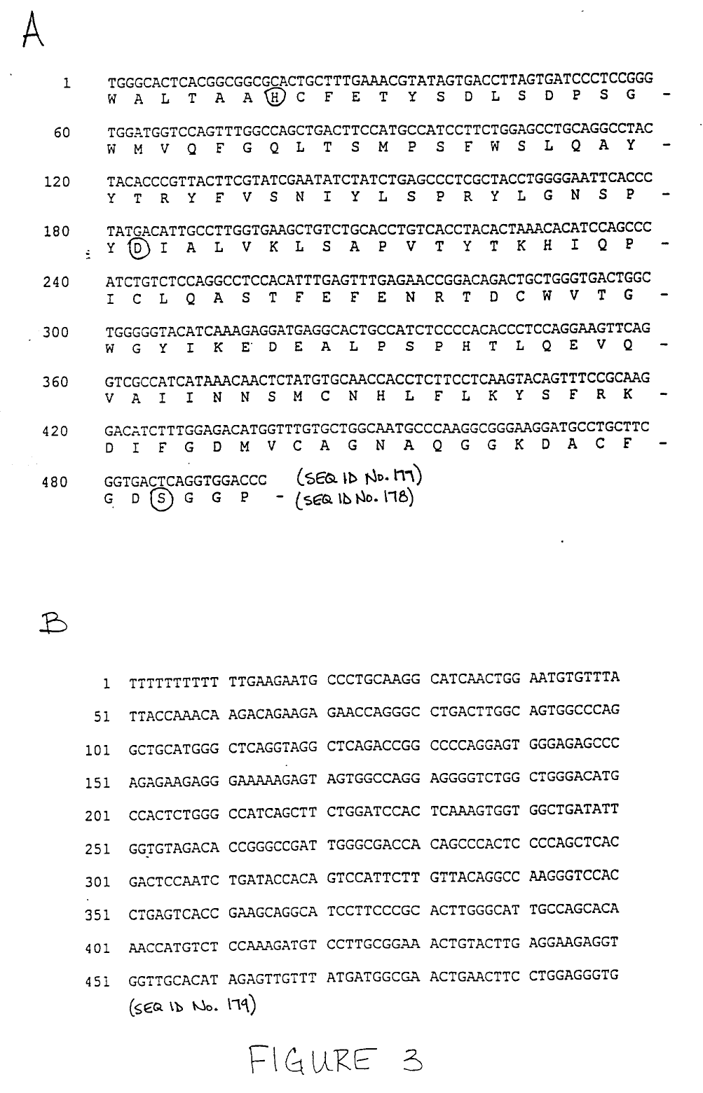

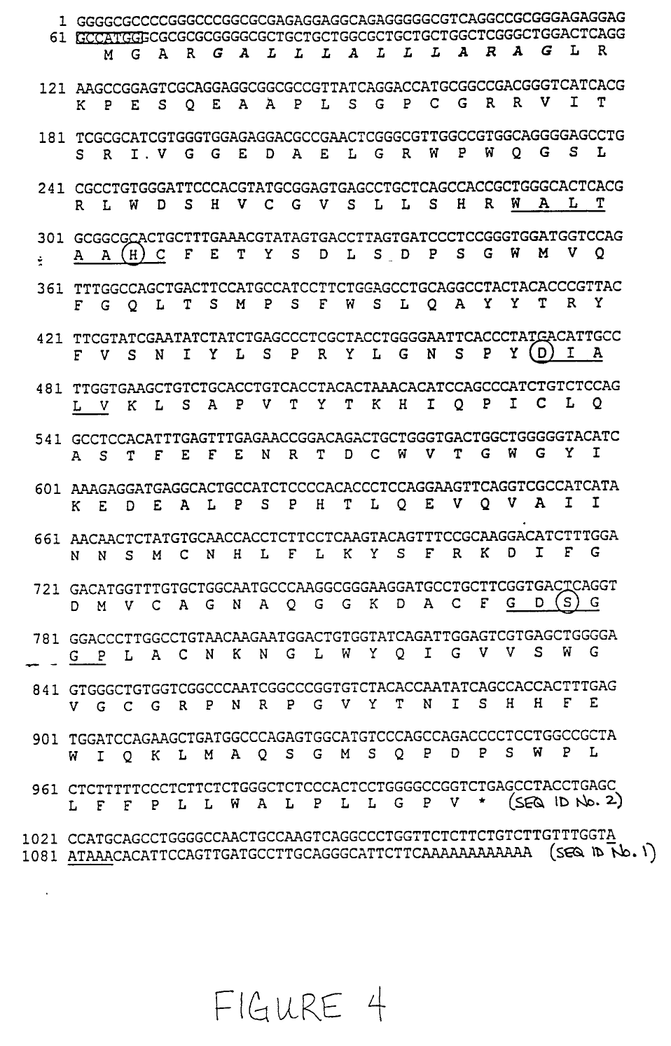

Comparison of the TADG-16 catalytic domain to the EST. database identified a homologous sequence (Accession No. AA620757) that overlapped a portion of the TADG-16 catalytic domain clone and also included the 3′-untranslated region and poly (A) tail of the TADG-16 transcript (FIG. 3). Comparison of the catalytic domain clone to the GenBank non-redundant database identified a genomic cosmid clone (Accession No. AC005361) homologous to the catalytic domain clone. Using the GRAIL exon identification program available through the National Center for Biotechnology Information, potential exons encoding the 5′ portion of the TADG-16 transcript were identified.

### EXAMPLE 4

Cloning of the TADG-16 cDNA

A sense PCR primer (T16-F1: 5′-GTCAGGCCGCGGGAGAGGAG-3′ (SEQ ID No. 10)) was designed to the cDNA predicted by the Grail program and used in conjunction with an antisense primer (T16-R2: 5′-ACTCTGGGCCATCAGCTTCT-3′ (SEQ ID No. 11)) designed to the overlapping EST that included the polyA+tail (GenBank Accession No. AA620757 encoding bases 614 to 1129 of TADG-16). Additional antisense primers were utilized in 5′-RACE experiments using a human testes cDNA library as template to identify the 40-most 5′ bases. The sequence of the 5′-RACE primers are as follows:

T16-R6: 5′-CGGAGGGATCACTAAGGTCACTATACGT-3′ (SEQ ID No.12); and
T16-R7: 5′-TATACGTTTCAAAGCAGTGCGCCGCCGT-3′ (SEQ ID No.13).

This allowed for the identification of the 1129 bases of the sequence reported herein. Within this 1129 bases, there is a Kozak's consensus sequence for the initiation of translation, an open reading frame encoding a 314 amino acid protein and a polyadenylation signal.

### EXAMPLE 5

Tissue-Specific Expression of TADG-16

Using a previously authenticated semi-quantitative PCR technique (Shigemasa et al., J. Soc. Gynecological Inv., 4, 95-102 (1997)), the expression level of the TADG-16 transcript was examined in normal ovarian tissue and ovarian tumor specimens. To do this, a TADG-16-specific PCR product was co-amplified with a PCR product for β-tubulin as an internal control. To amplify a 237 bp PCR product specific for TADG-16, the following primers were used:

| T16-F2: 5′-GGTCGCCATCATAAACAACT-3′ | (SEQ ID No. |
|---|---|
| | 14); and |
| T16-R2: 5′-ACTCTGGGCCATCAGCTTCT-3′ | (SEQ ID No. |
| | 15). |

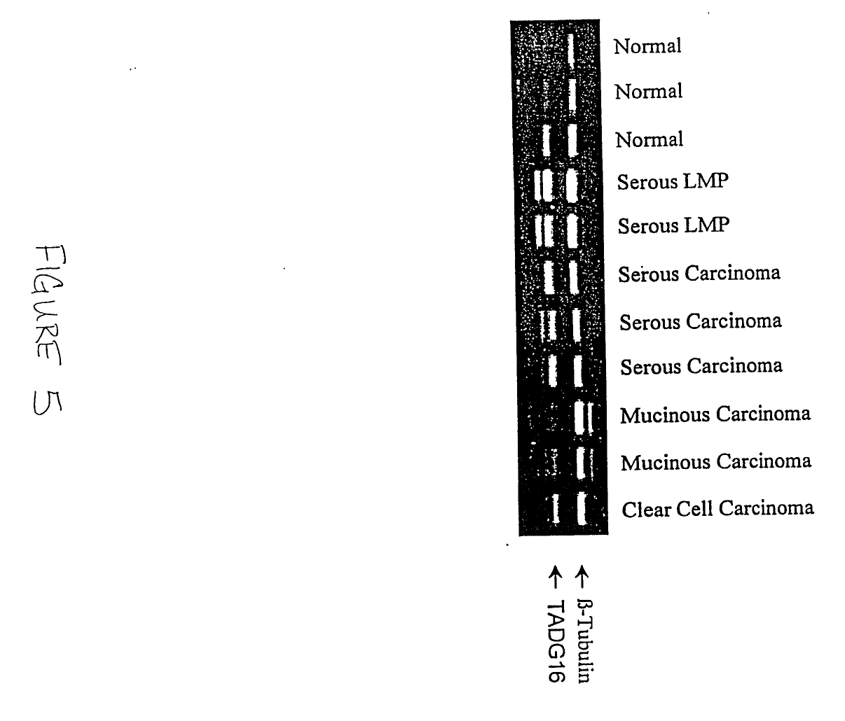

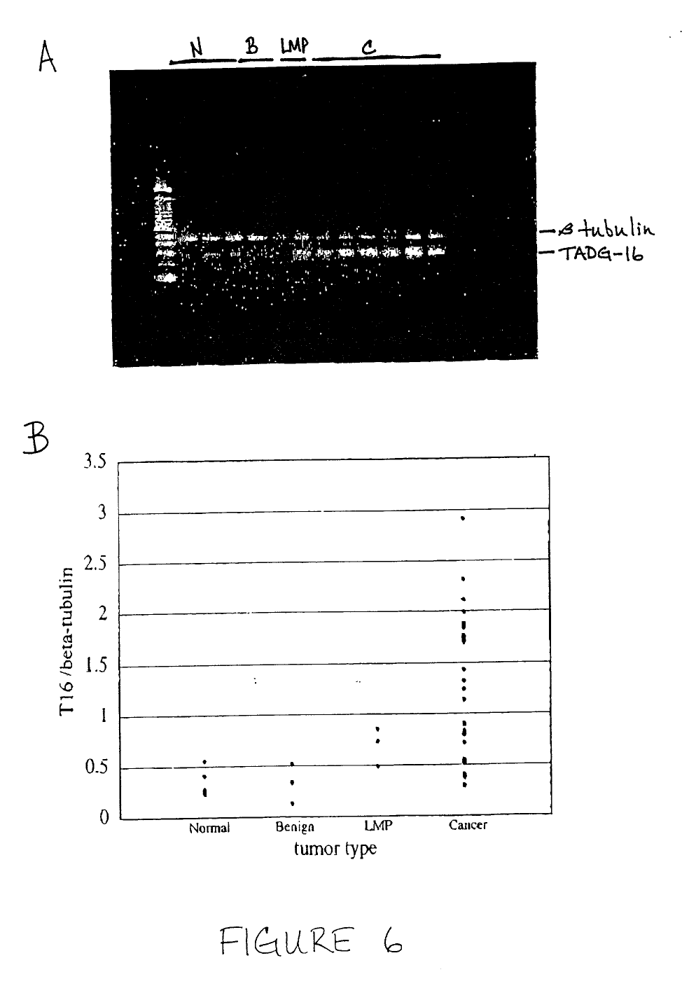

The reaction mixture was heated to 94° C. for 1.5 min, then 30 cycles of PCR was carried out under the following conditions: 30 sec of denaturation at 94° C., 30 sec of annealing at 62° C. and 30 sec of extension at 72° C. A final extension at 72° C. was performed for 7 min before the reaction was terminated. These PCR products were electrophoresed through an agarose gel to separate them based on size. Based on this experiment, TADG-16 appears to be expressed in tumor tissue (FIGS. 5 & 6).

### EXAMPLE 6

Expression of TADG-16 in Tumors

The expression of the serine protease TADG-16 gene in normal, low malignant potential tumors, and carcinoma (both mucinous and serous type) by quantitative PCR using TADG-16-specific primers was determined (primers directed toward the β-tubulin message were used as an internal standard). These data confirm the overexpression of the TADG-16 surface protease gene in ovarian carcinoma, including both low malignant potential tumors and overt carcinoma. Expression of TADG-16 is increased over normal levels in low malignant potential tumors, and high stage tumors (Stage III) of this group have higher expression of TADG-16 when compared to low stage tumors (Stage 1) (Table 3). In overt carcinoma, serous tumors exhibit the highest levels of TADG-16 expression, while mucinous tumors express levels of TADG-16 comparable with the high stage low malignant potential group.

TABLE 3
Expression of TADG-16

| Case No. | Code | Stage | Grade | Histology | TADG-16 |
|---|---|---|---|---|---|
| 1 | 1 | — | — | — | 0.553 |
| 2 | 1 | — | — | — | 0.232 |
| 3 | 1 | — | — | — | 0.229 |
| 4 | 1 | — | — | — | 0.400 |
| 5 | 1 | — | — | — | 0.226 |
| 6 | 1 | — | — | — | 0.230 |
| 7 | 1 | — | — | — | 0.269 |
| 8 | 2 | — | — | — | 0.121 |
| 9 | 2 | — | — | — | 0.514 |
| 10 | 2 | — | — | — | 0.333 |
| 11 | 2 | — | — | — | 0.323 |
| 12 | 3 | | | | 0.732 |
| 13 | 3 | | | | 0.487 |
| 14 | 3 | | | | 0.850 |
| 15 | 4 | 1 | 1 | 2 | 0.815 |
| 16 | 4 | 1 | 1 | 3 | 0.287 |
| 17 | 4 | 1 | 2 | 2 | 0.382 |
| 18 | 4 | 1 | 1 | 1 | 0.400 |
| 19 | 4 | 1 | 1 | 2 | 0.548 |
| 20 | 4 | 1 | 2 | 4 | 2.120 |
| 21 | 4 | 1 | 2 | 4 | 1.700 |
| 22 | 4 | 1 | 1 | 1 | 1.760 |
| 23 | 4 | 1 | 2 | 1 | 1.240 |
| 24 | 4 | 2 | 3 | 1 | 1.320 |
| 25 | 4 | 2 | 1 | 1 | 0.710 |
| 26 | 4 | 3 | 1 | 2 | 0.828 |
| 27 | 4 | 3 | 1 | 1 | 1.730 |
| 28 | 4 | 3 | 1 | 1 | 0.510 |
| 29 | 4 | 3 | 1 | 1 | 2.320 |
| 30 | 4 | 3 | 1 | 2 | 0.792 |
| 31 | 4 | 3 | 1 | 3 | 0.899 |
| 32 | 4 | 3 | 2 | 1 | 1.880 |
| 33 | 4 | 3 | 2 | 1 | 1.130 |
| 34 | 4 | 3 | 2 | 3 | 0.892 |
| 35 | 4 | 3 | 2 | 3 | 1.990 |
| 36 | 4 | 3 | 2 | 3 | 0.365 |
| 37 | 4 | 3 | 3 | 1 | 1.840 |
| 38 | 4 | 3 | 3 | 1 | 1.430 |
| 39 | 4 | 3 | 3 | 3 | 0.830 |
| 40 | 4 | 3 | 1 | 1 | 1.730 |
| 41 | 4 | 3 | 1 | 1 | 2.910 |

Code: 1, normal ovary; 2, benign tumor (adenoma); 3, LMP tumor; 4, cancer (adenocarcinoma).

Stage=Clinical stage: 1, stage 1; 2, stage 2; 3, stage 3.

Grade=Histological grade: 1, grade 1; 2, grade 2; 3, grade 3.

Histology: 1, serous carcinoma; 2, mucinous carcinoma; 3, endometrioid carcinoma; 4, clear cell carcinoma.

TABLE 4
mRNA Expression Levels of TADG-16 Gene in Ovarian Cancers

mRNA Expression Levels

| | N | mean | SD |
|---|---|---|---|
| Normal ovary | 7 | 0.306 | 0.126 |
| Benign tumor | 4 | 0.323 | 0.161 |
| LMP tumor | 3 | 0.690 | 0.185 |
| Ovarian cancer | 27 | 1.235 | 0.692 |
| Clinical stage | | | |
| Stage 1 | 9 | 1.028 | 0.695 |
| Stage 2 | 2 | 1.015 | 0.431 |
| Stage 3 | 16 | 1.380 | 0.711 |
| Histological grade | | | |
| Grade 1 | 14 | 1.160 | 0.794 |
| Grade 2 | 9 | 1.300 | 0.667 |
| Grade 3 | 4 | 1.355 | 0.415 |
| Histological type | | | |
| Serous | 14 | 1.494 | 0.688 |
| Mucinous | 5 | 0.673 | 0.199 |
| Endometrioid | 6 | 0.877 | 0.609 |
| Clear Cell | 2 | 1.910 | 0.297 |

TABLE 5

| | p-value |
|---|---|
| | (unpaired t-test) |
| Tumor type | |
| normal vs. benign | 0.8473 |
| normal vs. LMP | 0.0046 |
| normal vs. cancer | 0.0014 |
| benign vs. LMP | 0.0375 |
| benign vs. cancer | 0.0148 |
| LMP vs. cancer | 0.1905 |
| Stage | |
| stage 1 vs. stage 2 | 0.9808 |
| stage 1 vs. stage 3 | 0.2435 |
| stage 2 vs. stage 3 | 0.4951 |
| Grade | |
| grade 1 vs. grade 2 | 0.6659 |
| grade 1 vs. grade 3 | 0.6472 |
| grade 2 vs. grade 3 | 0.8830 |
| Histology | |
| serous vs. mucinous | 0.0192 |
| serous vs. endometrioid | 0.0743 |
| serous vs. clear cell | 0.4230 |
| mucinous vs. endometrioid | 0.4937 |
| mucinous vs. clear cell | 0.0012 |
| endometrioid vs. clear cell | 0.0678 |

### EXAMPLE 7

Antisense TADG-16

TADG-16 is cloned and expressed in the opposite orientation such that an antisense RNA molecule (SEQ ID No. 16) is produced. For example, the antisense RNA is used to hybridize to the complementary RNA in the cell and thereby inhibit translation of TADG-16 RNA into protein.

### EXAMPLE 8

Peptide Ranking

For vaccine or immune stimulation, individual 9-mers to 11-mers of the TADG-16 protein were examined to rank the binding of individual peptides to the top 8 haplotypes in the general population (Parker et al., (1994)). The computer program used for this analyses can be found at <http://www-bimas.dcrt.nih.gov/molbio/hla_bind/>. Table 6 shows the peptide ranking based upon the predicted half-life of each peptide's binding to a particular HLA allele. A larger half-life indicates a stronger association with that peptide and the particular HLA molecule. The TADG-16 peptides that strongly bind to an HLA allele are putative immunogens, and are used to innoculate an individual against hepsin.

TABLE 6
TADG-16 peptide ranking

| HLA Type | | | Predicted | SEQ |
|---|---|---|---|---|
| & Ranking | Start | Peptide | Dissociation_{1/2} | ID No. |
| HLA A0201 | | | | |
| 1 | 70 | SLLSHRWAL | 592.807 | 17 |
| 2 | 299 | LLFFPLLWA | 395.296 | 18 |
| 3 | 142 | KLSAPVTYT | 329.937 | 19 |
| 4 | 96 | WMVQFGQLT | 94.077 | 20 |
| 5 | 10 | ALLLARAGL | 79.041 | 21 |
| 6 | 252 | QIGVVSWGV | 71.726 | 22 |
| 7 | 248 | GLWYQIGVV | 70.769 | 23 |
| 8 | 139 | ALVKLSAPV | 69.552 | 24 |
| 9 | 291 | SQPDPSWPL | 66.602 | 25 |
| 10 | 130 | YLGNSPYDI | 47.991 | 26 |
| 11 | 190 | TLQBVQVAI | 42.774 | 27 |
| 12 | 6 | ALLLALLLA | 42.278 | 28 |
| 13 | 165 | FENRTDCWV | 34.216 | 29 |
| 14 | 71 | LLSHRWALT | 21.536 | 30 |
| 15 | 8 | LLALLLARA | 19.425 | 31 |
| 16 | 297 | WPLLFFPLL | 17.136 | 32 |
| 17 | 113 | QAYYTRYFV | 17.002 | 33 |
| 18 | 123 | NIYLSPRYL | 10.339 | 34 |
| 19 | 104 | TSMFSFWSL | 7.352 | 35 |
| 20 | 273 | NISHHFEWI | 7.345 | 36 |
| HLA A0205 | | | | |
| 1 | 70 | SLLSHRWAL | 25.200 | 37 |
| 2 | 42 | IVGGEDAEL | 23.800 | 38 |
| 3 | 10 | ALLLARAGL | 21.000 | 39 |
| 4 | 291 | SQPDPSWPL | 20.160 | 40 |
| 5 | 297 | WPLLFFPLL | 12.600 | 41 |
| 6 | 248 | GLWYQIGVV | 12.000 | 42 |
| 7 | 82 | HCFETYSDL | 6.300 | 43 |
| 8 | 142 | KLSAPVTYT | 6.000 | 44 |
| 9 | 96 | WMVQFGQLT | 6.000 | 45 |
| 10 | 299 | LLFFPLLWA | 5.100 | 46 |
| 11 | 303 | PLLWALPLL | 4.200 | 47 |
| 12 | 123 | NIYLSPRYL | 4.200 | 48 |
| 13 | 98 | VQFGQLTSM | 4.080 | 49 |
| 14 | 306 | WALPLLGPV | 3.600 | 50 |
| 15 | 71 | LLSHRWALT | 3.400 | 51 |
| 16 | 53 | WPWQGSLRL | 3.150 | 52 |
| 17 | 302 | FPLLWALPL | 3.150 | 53 |
| 18 | 130 | YLGNSPYDI | 3.000 | 54 |
| 19 | 6 | ALLLALLLA | 3.000 | 55 |
| 20 | 190 | TLQEVQVAI | 3.000 | 56 |
| HLA A1 | | | | |
| 1 | 44 | GGEDAELGR | 11.250 | 57 |
| 2 | 90 | LSDPSGWMV | 7.500 | 58 |
| 3 | 143 | LSAPVTYTK | 6.000 | 59 |
| 4 | 292 | QPDPSWPLL | 2.500 | 60 |
| 5 | 203 | MCNHLFLKY | 2.500 | 61 |
| 6 | 87 | YSDLSDPSG | 1.500 | 62 |
| 7 | 168 | RTDCWVTGW | 1.250 | 63 |
| 8 | 47 | DAELGRWPW | 0.900 | 64 |
| 9 | 23 | SQEAAPLSG | 0.675 | 65 |
| 10 | 7 | LLLALLLAR | 0.500 | 66 |
| 11 | 157 | CLQASTFEF | 0.500 | 67 |
| 12 | 202 | SMCNHLFLK | 0.500 | 68 |
| 13 | 111 | SLQAYYTRY | 0.500 | 69 |
| 14 | 125 | YLSPRYLGN | 0.500 | 70 |
| 15 | 152 | HIQPICLQA | 0.500 | 71 |
| 16 | 79 | TAAHCFETY | 0.500 | 72 |
| 17 | 238 | SGGPLACNK | 0.500 | 73 |
| 18 | 172 | WVTGWGYIK | 0.400 | 74 |
| 19 | 110 | WSLQAYYTR | 0.300 | 75 |
| 20 | 191 | LQEVQVAII | 0.270 | 76 |
| HLA A24 | | | | |
| 1 | 118 | RYFVSNIYL | 400.000 | 77 |
| 2 | 177 | GYIKEDEAL | 300.000 | 78 |
| 3 | 210 | KYSFRKDIF | 140.000 | 79 |
| 4 | 270 | VYTNISHHF | 60.000 | 80 |
| 5 | 148 | TYTKHIQPI | 28.800 | 81 |
| 6 | 300 | LFFPLLWAL | 24.000 | 82 |
| 7 | 234 | CFGDSGGPL | 22.000 | 83 |
| 8 | 135 | PYDIALVKL | 9.600 | 84 |
| 9 | 4 | RGALLLALL | 8.640 | 85 |
| 10 | 104 | TSMPSFWSL | 8.640 | 86 |
| 11 | 296 | SWPLLFFPL | 7.500 | 87 |
| 12 | 250 | WYQIGVVSW | 7.200 | 88 |
| 13 | 5 | GALLLALLL | 7.200 | 89 |
| 14 | 95 | GWMVQFGQL | 7.200 | 90 |
| 15 | 199 | INNSMCNHL | 7.200 | 91 |
| 16 | 297 | WPLLFFPLL | 7.200 | 92 |
| 17 | 291 | WQPDPSWPL | 7.200 | 93 |
| 18 | 183 | EALPSPHTL | 7.200 | 94 |
| 19 | 86 | TYSDLSDPS | 7.200 | 95 |
| 20 | 10 | ALLLARAGL | 6.000 | 96 |
| HLA B7 | | | | |
| 1 | 297 | WPLLFFPLL | 80.000 | 97 |
| 2 | 302 | FPLLWALPL | 80.000 | 98 |
| 3 | 53 | WPWQGSLRL | 80.000 | 99 |
| 4 | 292 | QPDPSWPLL | 24.000 | 100 |
| 5 | 145 | APVTYTKHI | 24.000 | 101 |
| 6 | 42 | IVGGEDAEL | 20.000 | 102 |
| 7 | 10 | ALLLARAGL | 18.000 | 103 |
| 8 | 104 | TSMPSFWSL | 12.000 | 104 |
| 9 | 183 | EALPSPHTL | 12.000 | 105 |
| 10 | 201 | NSMCNHLFL | 12.000 | 106 |
| 11 | 5 | GALLLALLL | 12.000 | 107 |
| 12 | 291 | SQPDPSWPL | 6.000 | 108 |
| 13 | 70 | SLLSHRWAL | 6.000 | 109 |
| 14 | 195 | QVAIINNSM | 5.000 | 110 |
| 15 | 116 | YTRYFVSNI | 4.000 | 111 |
| 16 | 199 | INNSMCNHL | 4.000 | 112 |
| 17 | 82 | HCFETYSDL | 4.000 | 113 |
| 18 | 132 | GNSPYDIAL | 4.000 | 114 |
| 19 | 1 | MGARGALLL | 4.000 | 115 |
| 20 | 63 | DSHVCGVSL | 4.000 | 116 |
| HLA B8 | | | | |
| 1 | 183 | EALPSPHTL | 1.600 | 117 |
| 2 | 58 | SLRLWDSHV | 1.200 | 118 |
| 3 | 82 | HCFETYSDL | 1.200 | 119 |
| 4 | 116 | YTRYFVSNI | 1.000 | 120 |
| 5 | 2 | GARGALLLA | 0.800 | 121 |
| 6 | 302 | FPLLWALPL | 0.800 | 122 |
| 7 | 53 | WPWQGSLRL | 0.800 | 123 |
| 8 | 31 | GPCGRRVIT | 0.800 | 124 |
| 9 | 297 | WPLLFFPLL | 0.800 | 125 |
| 10 | 5 | GALLLALLL | 0.800 | 126 |
| 11 | 71 | LLSHRWALT | 0.400 | 127 |
| 12 | 242 | LACNKNGLW | 0.400 | 128 |
| 13 | 10 | ALLLARAGL | 0.400 | 129 |
| 14 | 70 | SLLSHRWAL | 0.400 | 130 |
| 15 | 63 | DSHVCGVSL | 0.400 | 131 |
| 16 | 89 | DLSDPSGWM | 0.300 | 132 |
| 17 | 132 | GNSPYDIAL | 0.200 | 133 |
| 18 | 140 | LVKLSAPVT | 0.200 | 134 |
| 19 | 149 | YTKHIQPIC | 0.200 | 135 |
| 20 | 15 | RAGLRKPES | 0.200 | 136 |
| HLA B2702 | | | | |
| 1 | 117 | GRWPWQVSL | 1000.000 | 137 |
| 2 | 51 | LRSDQEPLY | 300.00 | 138 |
| 3 | 263 | RRKLPVDRI | 200.000 | 139 |
| 4 | 74 | SRWRVFAGA | 100.000 | 140 |
| 5 | 128 | GRDTSLGRW | 100.000 | 141 |
| 6 | 266 | WRLCGIVSW | 60.000 | 142 |
| 7 | 3 | LRYDGAHLC | 60.000 | 143 |
| 8 | 34 | LRALTHSEL | 60.000 | 144 |
| 9 | 213 | FREWIFQAI | 20.000 | 145 |
| 10 | 18 | GRLPHTQRL | 20.000 | 146 |
| 11 | 101 | ERNRVLSRW | 20.000 | 147 |
| 12 | 227 | NRVLSRWRV | 20.000 | 148 |
| 13 | 59 | SRPKVAALT | 20.000 | 149 |
| 14 | 40 | VRTAGANGT | 20.000 | 150 |
| 15 | 35 | QRLLEVISV | 18.000 | 151 |
| 16 | 98 | CQGDSGGPF | 10.000 | 152 |
| 17 | 112 | ARLMVFDKT | 6.000 | 153 |
| 18 | 291 | WRVFAGAVA | 6.000 | 154 |
| 19 | 191 | GRFLAAICQ | 6.000 | 155 |
| 20 | 157 | CLQASTFEF | 3.000 | 156 |
| HLA B4403 | | | | |
| 1 | 122 | SNIYLSPRY | 30.000 | 157 |
| 2 | 182 | DEALPSPHT | 24.000 | 158 |
| 3 | 45 | GEDAELGRW | 18.000 | 159 |
| 4 | 136 | YDIALVKLS | 11.250 | 160 |
| 5 | 170 | DCWVTGWGY | 9.000 | 161 |
| 6 | 243 | ACNKNGLWY | 6.000 | 162 |
| 7 | 163 | FEFENRTDC | 6.000 | 163 |
| 8 | 88 | SDLSDPSGW | 6.000 | 164 |
| 9 | 79 | TAAHCFETY | 6.000 | 165 |
| 10 | 278 | FEWIQKLMA | 6.000 | 166 |
| 11 | 192 | QEVQVAIIN | 5.400 | 167 |
| 12 | 92 | DPSGWMVQF | 4.500 | 168 |
| 13 | 294 | DPSWPLLFF | 4.500 | 169 |
| 14 | 203 | MCNHLFLKY | 4.500 | 170 |
| 15 | 76 | WALTAAHCF | 4.500 | 171 |
| 16 | 165 | FENRTDCWV | 4.000 | 172 |
| 17 | 215 | KDIFGDMVC | 2.500 | 173 |
| 18 | 48 | AELGRWPWQ | 2.400 | 174 |
| 19 | 272 | TNISHHFEW | 2.250 | 175 |
| 20 | 227 | AQGGKDACF | 2.250 | 176 |

Implications

That TADG-16 is found at low levels in some normal tissues may not detract from it's potential usefulness as a tumor marker, as there may be an aberrant expression pattern at the translational level that, e.g., allows for detection of TADG-16 in tumor patients but not in healthy patients, and/or activation of the TADG-16 enzyme may be necessary for tumor progression. In the case of the serine protease hepsin, Torres-Rosada et al. demonstrated by down-regulating hepsin that hepsin was required for growth of certain mammalian cells in culture.

The TADG-16 protein sequence is 314 amino acids in length and contains a secretion signal sequence, which suggests that this protein is functional in an extracellular capacity. A proteolytic cleavage site usually associated with protease enzyme activation is present downstream from the secretion signal sequence between amino acid residues 19 and 20. Moreover, the identified clone contains the necessary amino acids characteristic of a functional serine protease catalytic triad, thereby suggesting that this protein may be functioning in a manner that would promote cellular growth or expansion.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. DNA encoding a tumor antigen-derived gene (TADG-16) protein, selected from the group consisting of:
(a) isolated DNA which encodes a TADG-16 protein;
(b) isolated DNA which hybridizes under high stringency conditions to the isolated DNA of (a) above and which encodes a TADG-16 protein; and
(c) isolated DNA differing from the isolated DNAs of (a) and (b) above in codon sequence due to the degeneracy of the genetic code, and which encodes a TADG-16 protein.

2. The DNA of claim 1, wherein said DNA has the sequence shown in SEQ ID No. 1.

3. The DNA of claim 1, wherein said TADG-16 protein has the amino acid sequence shown in SEQ ID No. 2.

4. An oligonucleotide having the nucleotide sequence complementary to a sequence of claim 1.

5. A composition comprising the oligonucleotide according to claim 4 and a physiologically acceptable carrier therefore.

6. A vector comprising the DNA of claim 1 and regulatory elements necessary for expression of said DNA in a cell.

7. The vector of claim 6, wherein said DNA encodes a TADG-16 protein having the amino acid sequence shown in SEQ ID No. 2.

8. The vector of claim 6, wherein said DNA is positioned in reverse orientation relative to said regulatory elements such that TADG-16 antisense mRNA is produced.

9. A host cell transfected with the vector of claim 6 said vector expressing a TADG-16 protein.

10. The host cell of claim 9, wherein said cell is selected from the group consisting of bacterial cells, mammalian cells, plant cells and insect cells.

11. The host cell of claim 10, wherein said bacterial cell is E. coli.

12. Isolated and purified TADG-16 protein coded for by DNA selected from the group consisting of:
(a) isolated DNA which encodes a TADG-16 protein;
(b) isolated DNA which hybridizes under high stringency conditions to isolated DNA of (a) above and which encodes a TADG-16 protein; and
(c) isolated DNA differing from the isolated DNAs of (a) and (b) above in codon sequence due to the degeneracy of the genetic code, and which encodes a TADG-16 protein.

13. The TADG-16 protein of claim 12, wherein said protein has the amino acid sequence shown in SEQ ID No. 2.

14. An antibody, wherein said antibody is specific for TADG-16 protein or a fragment thereof.

15. A method for detecting TADG-16 mRNA in a sample, comprising the steps of:
(a) contacting a sample with a probe, wherein said probe is specific for TADG-16; and
(b) detecting binding of said probe to TADG-16 mRNA in said sample.

16. The method of claim 15, wherein said sample is a biological sample.

17. The method of claim 16, wherein said biological sample is from an individual.

18. The method of claim 17, wherein said individual is suspected of having cancer.

19. A kit for detecting TADG-16 mRNA, comprising:
an oligonucleotide probe, wherein said probe is specific for TADG-16.

20. The kit of claim 19, further comprising:
a label with which to label said probe; and
means for detecting said label.

21. A method of detecting TADG-16 protein in a sample, comprising the steps of:
(a) contacting a sample with an antibody, wherein said antibody is specific for TADG-16 or a fragment thereof; and
(b) detecting binding of said antibody to TADG-16 protein in said sample.

22. The method of claim 21, wherein said sample is a biological sample.

23. The method of claim 22, wherein said biological sample is from an individual.

24. The method of claim 23, wherein said individual is suspected of having cancer.

25. A kit for detecting TADG-16 protein, comprising:
an antibody, wherein said antibody is specific for TADG-16 protein or a fragment thereof.

26. The kit of claim 25, further comprising:
means to detect said antibody.

27. A method of inhibiting endogenous expression of TADG-16 in a cell, comprising the step of:
(a) introducing the vector of claim 8 into a cell, wherein expression of said vector produces TADG-16 antisense mRNA in said cell, wherein said TADG-16 antisense mRNA hybridizes to endogenous TADG-16 mRNA, thereby inhibiting endogenous expression of TADG-16 in said cell.

28. A method of inhibiting a TADG-16 protein in a cell, comprising the step of:
introducing an antibody into a cell, wherein said antibody is specific for a TADG-16 protein or a fragment thereof, wherein binding of said antibody to said TADG-16 protein inhibits said TADG-16 protein.

29. A method of treating a neoplastic state in an individual in need of such treatment, comprising the step of administering to said individual an effective dose of the oligonucleotide of claim 4.

30. The method of claim 29, wherein said neoplastic state is selected from the group consisting of ovarian cancer, breast cancer, lung cancer, colon cancer and prostate cancer.

31. A method of vaccinating an individual against TADG-16, comprising the steps of:
inoculating an individual with a TADG-16 protein or fragment thereof, wherein said TADG-16 protein or fragment thereof lack TADG-16 protease activity, wherein said inoculation with said TADG-16 protein or fragment thereof elicits an immune response in said individual, thereby vaccinating said individual against TADG-16.

32. The method of claim 31, wherein said TADG-16 fragment is selected from the group consisting of a 9-residue fragment up to a 20-residue fragment.

33. The method of claim 32, wherein said 9-residue fragment is selected from the group consisting of SEQ ID Nos. 17, 18, 19, 77, 78, 79, 80, 97, 98, 99, 137, 138, 139, 140 and 141.

34. The method of claim 31, wherein said individual has cancer, is suspected of having cancer or is at risk of getting cancer.

35. An immunogenic composition, comprising a n immunogenic fragment of a TADG-16 protein and an adjuvant.

36. The immunogenic composition of claim 35, wherein said fragment is selected from the group consisting of a 9-residue fragment up to a 20-residue fragment.

37. The immunogenic composition of claim 36, wherein said 9-residue fragment is selected from the group consisting of SEQ ID Nos. 17, 18, 19, 77, 78, 79, 80, 97, 98, 99, 137, 138, 139, 140 and 141.

38. A method of diagnosing cancer in an individual, comprising the steps of:
(a) obtaining a biological sample from an individual;
(b) detecting TADG-16 in said sample, wherein the presence of TADG-16 in said sample is indicative of the presence of carcinoma in said individual, wherein the absence of TADG-16 in said sample is indicative of the absence of carcinoma in said individual.

39. The method of claim 38, wherein said biological sample is selected from the group consisting of blood, urine, saliva, tears, interstitial

40. The method of claim 38, wherein said detection of said TADG-16 is by means selected from the group consisting of Northern blot, Western blot, PCR, dot blot, ELIZA sandwich assay, radioimmunoassay, DNA array chips and flow cytometry of tumor cells, wherein said tumor cells are labeled.

41. The method of claim 38, wherein said carcinoma is selected from the group consisting of ovarian, breast, lung, colon, prostate and other in which TADG-16 is overexpressed.

42. A method of screening for compounds that inhibit TADG-16, comprising the steps of:
(a) contacting a sample with a compound, wherein said sample comprises TADG-16 protein; and
(b) assaying for TADG-16 protease activity, wherein a decrease in said TADG-16 protease activity in the presence of said compound relative to TADG-16 protease activity in the absence of said compound is indicative of a compound that inhibits TADG-16.

43. A method of targeted therapy to an individual, comprising the step of:
administering a compound to an individual, wherein said compound has a targeting moiety and a therapeutic moiety, wherein said targeting moiety is specific for TADG-16.

44. The method of claim 43, wherein said targeting moiety is selected from the group consisting of an antibody specific for TADG-16 and a ligand that binds TADG-16 or a ligand binding domain thereof.

45. The method of claim 43, wherein said therapeutic moiety is selected from the group consisting of a radioisotope, a toxin, a chemotherapeutic agent, an immune stimulant and a cytotoxic agent.

46. The method of claim 43, wherein said individual suffers from a cancer selected from the group consisting of ovarian, lung, prostate, colon and others in which TADG-16 is overexpressed.
